(19) United States
(12) Patent Application Publication
(10) Pub. No.: US 2023/0114033 A1
(43) Pub. Date: Apr. 13, 2023

(54) Systems, Methods and Media for Intrapartum Prediction of Unfavorable Labor Outcomes

(71) Applicant: Mayo Foundation for Medical Education and Research, Rochester, MN (US)

(72) Inventors: Sherif A. Shazly, Rochester, MN (US); Abimbola O. Famuyide, Rochester, MN (US)

(21) Appl. No.: 17/911,156

(22) PCT Filed: Mar. 22, 2021
(86) PCT No.: PCT/US2021/023427

§ 371 Date: Sep. 13, 2022

Related U.S. Application Data
(60) Provisional application No. 62/994,084, filed on Mar. 24, 2020.

(51) Int. Cl. G16H 50/30; G06N 20/00

(52) U.S. Cl. CPC G16H 50/30; G06N 20/00

(57) ABSTRACT

In accordance with some embodiments, systems, methods, and media for intrapartum prediction of unfavorable labor outcomes are provided. In some embodiments, a system comprises a processor programmed to: generate a feature vector including static variables knowable when the patient goes into labor, and dynamic variables including a recent cervical dilation; provide the feature vector to a machine learning model trained using labeled feature vectors associated with patients with known labor outcomes, each labeled vector including static and dynamic variables including cervical dilation in the same range as the patients, and each labeled feature vector indicating whether one or more unfavorable outcomes was experienced; receive, from the model, a risk the patient will experience an unfavorable outcome; and cause information indicative of that risk to be presented to aid the user in determining whether to recommend intrapartum Cesarean delivery for the patient.

### CROSS-REFERENCE TO RELATED APPLICATIONS

This application is based on, claims the benefit of, and claims priority to, U.S. Provisional Application No. 62/994,084, filed Mar. 24, 2020, which is hereby incorporated herein by reference in its entirety for all purposes.

### STATEMENT REGARDING FEDERALLY SPONSORED RESEARCH

N/A

### BACKGROUND

Management recommendations of labor and delivery have evolved constantly to accommodate evidence from the literature. A major conundrum every obstetrician faces in managing laboring women is to weigh maternal and neonatal risks of delayed intervention against risks of unindicated Cesarean delivery (CD). Although the incidence of CD has substantially increased in the past three decades there has been no discernible decline in maternal or neonatal adverse outcomes, indicating that many of the decisions to complete delivery via CD or vaginally are not having the intended effect of reducing adverse outcomes.

Medical practitioners managing laboring women often lack the tools and information necessary to accurately determine when intervention via CD would be a net benefit. For example, while labor dystocia represents the most common indication for primary CD, the diagnosis of labor dystocia lacks a consistent evidence-based and globally acceptable definition. This may contribute, in part, to the increases in rates of CDs, without a corresponding decrease in adverse outcomes.

One of the earliest trials to define normal labor progress was conducted in 1955 by Friedman. Based on observations of 500 women in labor, Friedman described the normal course of labor, which became known as the “Friedman curve.” For several decades, the Friedman sloping curve had been cited as a reference of normal labor progress. The terms “latent labor” and “active labor” were introduced to the literature to discriminate initial slow interval (less than 3-3.5 cm) from subsequent accelerated labor course. In 1972, Philpott and Castle proposed the use of “alert lines” and “action lines” to facilitate management of labor through a prospective study of 624 Rhodesian African primigravidas and provided simplified directions to midwives in isolated areas.

The World Health Organization (WHO) relied on Friedman's data and these studies and has served as an important tool in managing labor, especially in low resource countries. The WHO labor partogram tool for managing labor and labor dystocia has been widely used, especially in low resource countries. Although WHO partogram has been adopted globally to standardize labor care and prevent prolonged labor, the routine use of the WHO partogram has been questioned. For example, a Cochrane review of three clinical trials (1,813 patients) which compared partogram to no partogram use did not show differences in CD rates, duration of first stage of labor, or rates of Apgar score less than 7 at 5 minutes. Despite the use of the WHO partogram, the rate of CD has substantially increased in the last three decades, reaching 32% of total deliveries in the United States in 2017. This trend has not been associated with concomitant decline in maternal or neonatal mortality. Furthermore, current rates of NICU admission among neonates delivered at term is notable, accounting for 4.6% of neonates delivered electively at 39 weeks of gestation. This trend is increasing and term neonates weighing at least 2500 grams at birth may represent more than 50% of NICU admissions.

In 2002, Zhang et al. studied 1,329 term nulliparous parturients and suggested that the Friedman curve may not be reflective of contemporary labor progress patterns in the study population. Zhang et al. hypothesized that current recommendations of management of labor, which were based on Friedman's study in the 1950s, may not be appropriate for current populations. A new partogram developed by Zhang et al. differs from the WHO partogram. For example, the 95^{th} percentile line, which corresponds to WHO action line, is an exponential-like stair line, which outlines contemporary course of cervical dilation. Unlike the WHO partogram which aims to prevent prolonged labor, Zhang et al. proposed using the new partogram as a clinical tool to prevent premature cesarean delivery, but did not take into account important maternal and neonatal outcomes. Thus, a secondary analysis of a prospective cohort study of 7,845 women with term low risk pregnancy from 2010 through 2014 was conducted to assess maternal and neonatal outcomes after implementation of the Zhang et al. partogram. Rosenbloom et al. reported that primary CD rate did not decline between 2010 and 2014 (15.8% vs. 17.7%, P 0.5). In addition, maternal and neonatal morbidity significantly increased in the same time frame in the study population. A multicenter, cluster-randomized controlled trial (LaPS trial) was conducted in 14 clusters in Norway; 7 obstetric units were randomly assigned to intervention (managed by Zhang's guidelines, n=3,972) versus 7 units that were assigned to control (managed by WHO partogram, n=3,305). Again, the rate of intrapartum CD was not significantly different between the 2 groups.

While these approaches attempt to decrease the incidence of adverse outcomes, the data shows that they are ineffective at achieving this goal. The reasons may be that these existing techniques for managing labor progression are based on traditional statistical approaches, which may tend to make unrealistic assumptions regarding the functional form of the model and distribution of variables. These assumptions are often not applicable in complex clinical situations such as the dynamic labor process. As a result, the models may not fit the data well and may not be generalizable.

Accordingly, new systems, methods, and media for intrapartum prediction of unfavorable labor outcomes are desirable.

### SUMMARY

In accordance with some embodiments of the disclosed subject matter, systems, methods, and media for intrapartum prediction of unfavorable labor outcomes are provided.

In accordance with some embodiments of the disclosed subject matter, a system for predicting a risk of one or more unfavorable labor outcomes in a patient is provided, the system comprising: at least one hardware processor that is programmed to: generate a feature vector that includes a first plurality of values and a second plurality of values, wherein the first plurality of values corresponds to a respective plurality of static variables that are knowable at a time a patient goes into labor, and the second plurality of values corresponds to a respective plurality of dynamic variables that are associated with a particular time during labor, the second plurality of values includes at least a most recent cervical dilation value; provide the feature vector to a trained machine learning model, wherein the trained machine learning model was trained using a plurality of labeled feature vectors associated with a respective plurality of patients associated with one or more known labor outcomes, wherein each of the plurality of labeled feature vectors included values corresponding to the plurality of static variables and the plurality of dynamic variables associated with a respective patient and associated with a cervical dilation value in a range that includes the most recent cervical dilation value, and each of the plurality of labeled feature vectors is associated with an indication of one or more unfavorable outcomes experienced by the respective patient; receive, from the trained machine learning model, an output indicative of a risk that the patient will experience at least one of the one or more unfavorable outcomes; and cause information indicative of the risk to be presented to a user to aid the user in determining whether to recommend intrapartum Cesarean delivery for the patient.

In some embodiments, the trained machine learning model is a gradient boosting machine model comprising a plurality of decision trees.

In some embodiments, the at least one hardware processor is further programmed to: receive, from a baseline machine learning model, a baseline output indicative of a risk that the patient will experience at least one of the one or more unfavorable outcomes based on variables knowable at the time the patient went into labor, wherein the baseline machine learning model was trained using a second plurality of labeled feature vectors associated with a respective plurality of patients associated with one or more known labor outcomes, wherein each of the second plurality of labeled feature vectors included values corresponding to the plurality of static variables associated with a respective patient and omitted any dynamic variables associated with the respective patient, and each of the plurality of labeled feature vectors is associated with an indication of one or more unfavorable outcomes experienced by the respective patient; and include the baseline output in the feature vector.

In some embodiments, the trained machine learning model is trained to predict risk that the patient will experience at least one of the one or more unfavorable outcomes based on data collected through 4 centimeters (cm) cervical dilation, and wherein the most recent cervical dilation value is a cervical dilation value that is at least 4 cm cervical dilation and less than 5 cm.

In some embodiments, the plurality of static variables includes variables corresponding to parity, a binary indication of whether the patient has previously delivered via Cesarean, and the patient's age.

In some embodiments, the plurality of dynamic variables includes variables corresponding to cervical dilation, cervical effacement, and head station.

In some embodiments, the at least one hardware processor is further programmed to: plot the outcome on a graph, wherein the graph includes a curve representing average risk scores for patients that did not experience unfavorable outcomes and a second curve representing risk scores for patients that experienced one or more unfavorable outcomes; and cause the graph to be presented as the information indicative of the risk.

In some embodiments, the at least one hardware processor is further programmed to: generate a second feature vector that includes the first plurality of values and a third plurality of values, wherein the third plurality of values corresponds to a respective plurality of dynamic variables that are associated with a second particular time during labor, including at least a most recent cervical dilation value that exceeds an upper limit of the range associated with the trained model; provide the second feature vector to a second trained machine learning model, wherein the second machine learning model was trained using a second plurality of labeled feature vectors associated with the respective plurality of patients associated with the one or more known labor outcomes, wherein each of the second plurality of labeled feature vectors included values corresponding to the plurality of static variables and the plurality of dynamic variables associated with a respective patient and associated with a cervical dilation value in a second range that includes the most recent cervical dilation value included in the third plurality of values, and each of the second plurality of labeled feature vectors is associated with an indication of one or more unfavorable outcomes experienced by the respective patient; receive, from the second trained machine learning model, a second output indicative of an updated risk that the patient will experience at least one of the one or more unfavorable outcomes; generate an updated graph by plotting the second outcome on the graph; and cause the updated graph to be presented to the user to aid the user in determining whether to recommend intrapartum Cesarean delivery for the patient.

In some embodiments, the range associated with the trained machine learning model includes cervical dilation from about 4 cm to less than 5 cm and the second range associated with the second trained machine learning model includes cervical dilation from about 5 cm to less than 6 cm.

In some embodiments, a method for predicting a risk of one or more unfavorable labor outcomes in a patient is provided, the method comprising: generating a feature vector that includes a first plurality of values and a second plurality of values, wherein the first plurality of values corresponds to a respective plurality of static variables that are knowable at a time a patient goes into labor, and the second plurality of values corresponds to a respective plurality of dynamic variables that are associated with a particular time during labor, the second plurality of values includes at least a most recent cervical dilation value; providing the feature vector to a trained machine learning model, wherein the trained machine learning model was trained using a plurality of labeled feature vectors associated with a respective plurality of patients associated with one or more known labor outcomes, wherein each of the plurality of labeled feature vectors included values corresponding to the plurality of static variables and the plurality of dynamic variables associated with a respective patient and associated with a cervical dilation value in a range that includes the most recent cervical dilation value, and each of the plurality of labeled feature vectors is associated with an indication of one or more unfavorable outcomes experienced by the respective patient; receiving, from the trained machine learning model, an output indicative of a risk that the patient will experience at least one of the one or more unfavorable outcomes; and causing information indicative of the risk to be presented to a user to aid the user in determining whether to recommend intrapartum Cesarean delivery for the patient.

In some embodiments, a non-transitory computer readable medium containing computer executable instructions that, when executed by a processor, cause the processor to perform a method for predicting a risk of one or more unfavorable labor outcomes in a patient, the method comprising: generating a feature vector that includes a first plurality of values and a second plurality of values, wherein the first plurality of values corresponds to a respective plurality of static variables that are knowable at a time a patient goes into labor, and the second plurality of values corresponds to a respective plurality of dynamic variables that are associated with a particular time during labor, the second plurality of values includes at least a most recent cervical dilation value; providing the feature vector to a trained machine learning model, wherein the trained machine learning model was trained using a plurality of labeled feature vectors associated with a respective plurality of patients associated with one or more known labor outcomes, wherein each of the plurality of labeled feature vectors included values corresponding to the plurality of static variables and the plurality of dynamic variables associated with a respective patient and associated with a cervical dilation value in a range that includes the most recent cervical dilation value, and each of the plurality of labeled feature vectors is associated with an indication of one or more unfavorable outcomes experienced by the respective patient; receiving, from the trained machine learning model, an output indicative of a risk that the patient will experience at least one of the one or more unfavorable outcomes; and causing information indicative of the risk to be presented to a user to aid the user in determining whether to recommend intrapartum Cesarean delivery for the patient.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of a system for intrapartum prediction of unfavorable labor outcomes in accordance with some embodiments of the disclosed subject matter.

FIG. 2 shows an example of hardware that can be used to implement a computing device, and a server, shown in FIG. 1 in accordance with some embodiments of the disclosed subject matter.

FIG. 3 shows an example of a group of models that can be used to predict intrapartum risk of unfavorable labor outcomes over time as dynamic variables change in accordance with some embodiments of the disclosed subject matter.

FIG. 4 shows an example of a flow for training and using mechanisms for intrapartum prediction of unfavorable labor outcomes in accordance with some embodiments of the disclosed subject matter.

FIG. 5 shows an example of a process for training a machine learning model to predict intrapartum risks of unfavorable labor outcomes in accordance with some embodiments of the disclosed subject matter.

FIG. 6 shows an example of a process for using a machine learning model to predict intrapartum risk of unfavorable labor outcomes in accordance with some embodiments of the disclosed subject matter.

FIG. 7A shows an example of trends in average risk scores over labor progress for deliveries that were complicated (“YES”) and not complicated (“NO”) predicted by machine learning models implemented in accordance with some embodiments of the disclosed subject matter.

FIG. 7B shows an example of trends in average risk scores over labor progress for intrapartum Cesarean delivery (“YES”) and vaginal deliveries (“NO”) predicted by machine learning models implemented in accordance with some embodiments of the disclosed subject matter.

FIG. 8A shows an example of variables that had the largest impact on the predicted risk score for a machine learning model implemented in accordance with some embodiments of the disclosed subject matter to predict a risk of a composite of unfavorable labor outcomes based on variables measurable at admission.

FIG. 8B shows an example of variables that had the largest impact on the predicted risk score for a machine learning model implemented in accordance with some embodiments of the disclosed subject matter to predict a risk of a composite of unfavorable labor outcomes based on variables measurable up to 4 cm cervical dilation.

FIG. 8C shows an example of variables that had the largest impact on the predicted risk score for a machine learning model implemented in accordance with some embodiments of the disclosed subject matter to predict a risk of a composite of unfavorable labor outcomes based on variables measurable up to 6 cm dilation.

FIG. 8D shows an example of variables that had the largest impact on the predicted risk score for a machine learning model implemented in accordance with some embodiments of the disclosed subject matter to predict a risk of a composite of unfavorable labor outcomes based on variables measurable up to 8 cm dilation.

FIG. 8E shows an example of variables that had the largest impact on the predicted risk score for a machine learning model implemented in accordance with some embodiments of the disclosed subject matter to predict a risk of a composite of unfavorable labor outcomes based on variables measurable up to 10 cm dilation.

FIG. 9A shows an example of performance of a machine learning model implemented in accordance with some embodiments of the disclosed subject matter to predict a risk of a composite of unfavorable labor outcomes based on variables measurable at admission.

FIG. 9B shows an example of performance of a machine learning model implemented in accordance with some embodiments of the disclosed subject matter to predict a risk of a composite of unfavorable labor outcomes based on variables measurable at up to 4 cm cervical dilation.

FIG. 9C shows an example of performance of a machine learning model implemented in accordance with some embodiments of the disclosed subject matter to predict a risk of a composite of unfavorable labor outcomes based on variables measurable at up to 6 cm cervical dilation.

FIG. 9D shows an example of performance of a machine learning model implemented in accordance with some embodiments of the disclosed subject matter to predict a risk of a composite of unfavorable labor outcomes based on variables measurable at up to 8 cm cervical dilation.

FIG. 9E shows an example of performance of a machine learning model implemented in accordance with some embodiments of the disclosed subject matter to predict a risk of a composite of unfavorable labor outcomes based on variables measurable at up to 10 cm cervical dilation.

### DETAILED DESCRIPTION

In accordance with various embodiments, mechanisms (which can, for example, include systems, methods, and media) for intrapartum prediction of unfavorable labor outcomes are provided.

In accordance with some embodiments of the disclosed subject matter, mechanisms described herein can be used to establish an individualized labor chart, through a series of intrapartum prediction models using machine learning techniques that incorporate data on CD and obstetric outcomes. In some embodiments, mechanisms described herein can facilitate patient counseling and decision making and reduce the rate of CD, maternal complications, and neonatal complications.

In some embodiments of the disclosed subject matter, mechanisms described herein can train a series of intrapartum models that use baseline variables and dynamic (intrapartum) variables to predict the probability of unfavorable labor outcome (sometimes referred to herein as a Labor Risk Score; LRS). An unfavorable labor outcome can be one of the following: unsuccessful vaginal delivery (e.g., leading to CD in active labor), postpartum hemorrhage (e.g., defined as estimated blood loss that is greater than 1000 ml) or need for transfusion of blood products, suspected or confirmed intra-amniotic infection (IAI), shoulder dystocia, neonatal admission to intensive care unit (NICU), an APGAR score below 7 at 5 minutes, an umbilical arterial pH below 7.00, neonatal hypoxemic ischemic encephalopathy (HIE), neonatal ventilation use or continuous positive airway pressure (CPAP) therapy, neonatal intracranial hemorrhage (ICH), neonatal sepsis, or neonatal death. In some embodiments, the mechanisms described herein can generate an LRS that is indicative of a probability of an unfavorable labor outcome which can be defined as a composite of the preceding unfavorable outcomes.

In some embodiments, mechanisms described herein can train a set of prediction models to predict the primary outcome (e.g., LRS). For example, mechanisms described herein can be used to train a baseline model using variables identified at the time of patient admission (baseline predictors), and can be used to train a series of intrapartum prediction models that incorporate dynamic variables (e.g., which are determined by pelvic examination starting at cervical dilation of 4 cm) and other variables, such as whether oxytocin was used to augment labor, and whether meconium stained amniotic fluid was observed. Examples of particular dynamic variables can include current cervical dilation (e.g., in centimeters (cm)), cervical effacement (e.g., categorized as 0-30%, 40-50%, 60-70%, 80 or more), head station (e.g., categorized as −3, −2, −1 or 0, +1 or +2), time interval elapsed between a current examination and the immediately preceding examination, change in cervical dilation between a current examination and the immediately preceding examination, fetal heart rate, and dilation delta (e.g., defined as a change in cervical dilation from the immediately preceding examination divided by the time interval between the two examinations). In some embodiments, intrapartum variables that could not be linked to a particular cervical dilation in the training data (e.g., the presence of meconium stained amniotic fluid) can be incorporated in the 10 cm prediction model. However, this is merely an example, and such variables can be incorporated into a different model, or multiple models. Note that although intrapartum fetal heart rate may provide valuable predictive information, it is not described in the examples below as information on intrapartum fetal rate monitoring lacks documentation in the “Consortium on Safe Labor” database.

In some embodiments, mechanisms described herein can train the baseline prediction model to estimate the probability of an unfavorable primary outcome (e.g., LRS) based on the baseline variables only, and can train each intrapartum prediction model to estimate the probability of an unfavorable primary outcome (e.g., LRS) based on the baseline variables and dynamic labor variables. Additionally, in some embodiments, mechanisms described herein can train intrapartum prediction model corresponding to later stages of labor (e.g., larger cervical dilation) based on the most recent output from a trained baseline prediction model and/or one or more trained intrapartum prediction models corresponding to an earlier stage(s) of labor (e.g., a smaller cervical dilation). For example, mechanisms described herein can train an intrapartum prediction model to estimate the probability of an unfavorable primary outcome at 6 cm cervical dilation based on baseline variables, dynamic variables, and an output of an intrapartum prediction model that has already been trained to estimate the probability of an unfavorable primary outcome at 5 cm cervical dilation.

In some embodiments, mechanisms described herein can use one or more techniques to adjust dynamic confounders in data that is needed to predict maternal and neonatal outcomes more accurately. For example, because the progress of labor is affected by time-varying (or dynamic) confounders, such techniques for appropriately adjusting such dynamic confounders can be used to predict maternal and neonatal outcomes more accurately. Zhang et al. adopted methods that were limited in capturing this dynamic aspect of the data, which may be in part what led to the Zhang partogram being less successful than anticipated in reducing adverse outcomes. In some embodiments, mechanisms described herein can use machine learning techniques to incorporate representative features from changing labor characteristics into a trained machine learning model that can more successfully account for such dynamic confounders.

Existing analytic techniques for predicting labor progression have been based on traditional statistical approaches, which tend to make unrealistic assumptions regarding the functional form of the model and distribution of variables. These assumptions are often not applicable in complex clinical situations such as the dynamic labor process. As a result, the models may not fit the data well and may not be generalizable. Machine learning techniques can estimate complex relationships between clinical measurements with reasonable accuracy, thus producing robust and consistent estimates, without making a priori assumptions. In some embodiments, mechanisms described herein can use machine learning techniques to collectively analyze patterns of changes in usual prenatal and intrapartum variables based on the Data and Specimen Hub (DASH) database produced by the Eunice Kennedy Shriver National Institute of Child Health and Human Development (NICHD). In some embodiments, mechanisms described herein can be used to train an incremental gradient boosting machine (GBM)-based model, which can start from a baseline model that relies on variables that are available at admission. Note that static variables are generally described as being available at admission, this is merely an example, and these variables can be thought of as variables that are knowable at the time that the patient goes into labor whether the patient has been admitted at that time or ever will be admitted to a medical facility (e.g., mechanisms described herein can be used in connection with a patient that is attempting to delivery outside of a medical facility) In some embodiments, dynamic labor variables (e.g., at cervical dilation [4, 10] cm) can be incrementally used to extend the knowledge of an existing GBM model.

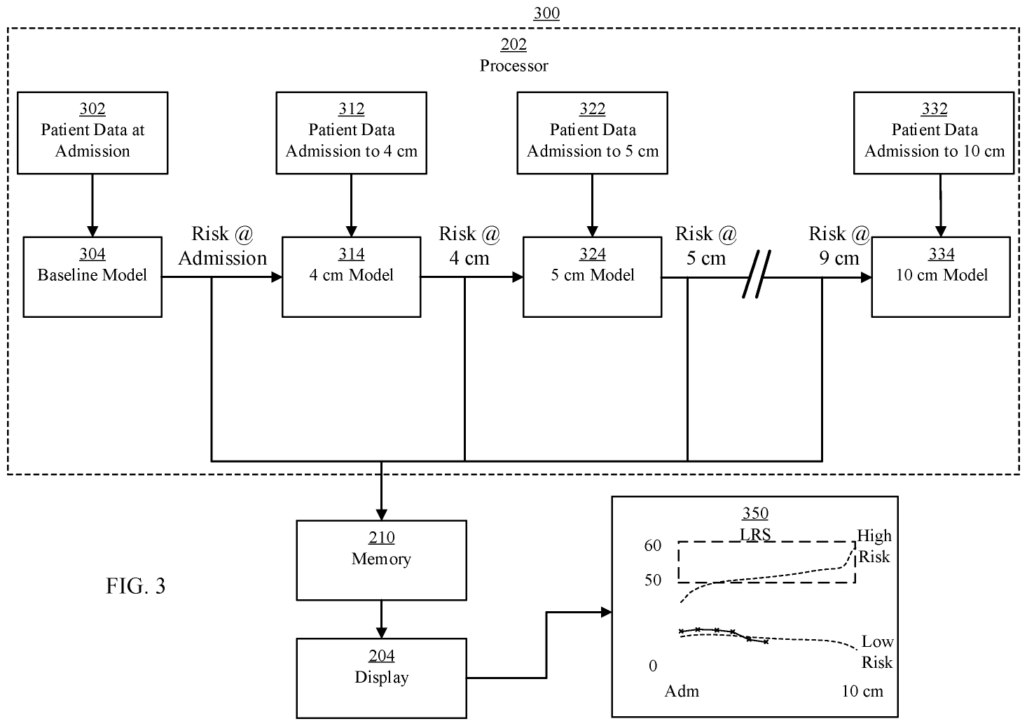

Note that while FIG. 3 depicts models being executed by processor 202, this is merely an example, and models can be executed by any suitable processor or combination of processors, such as processor 202 and/or processor 212.

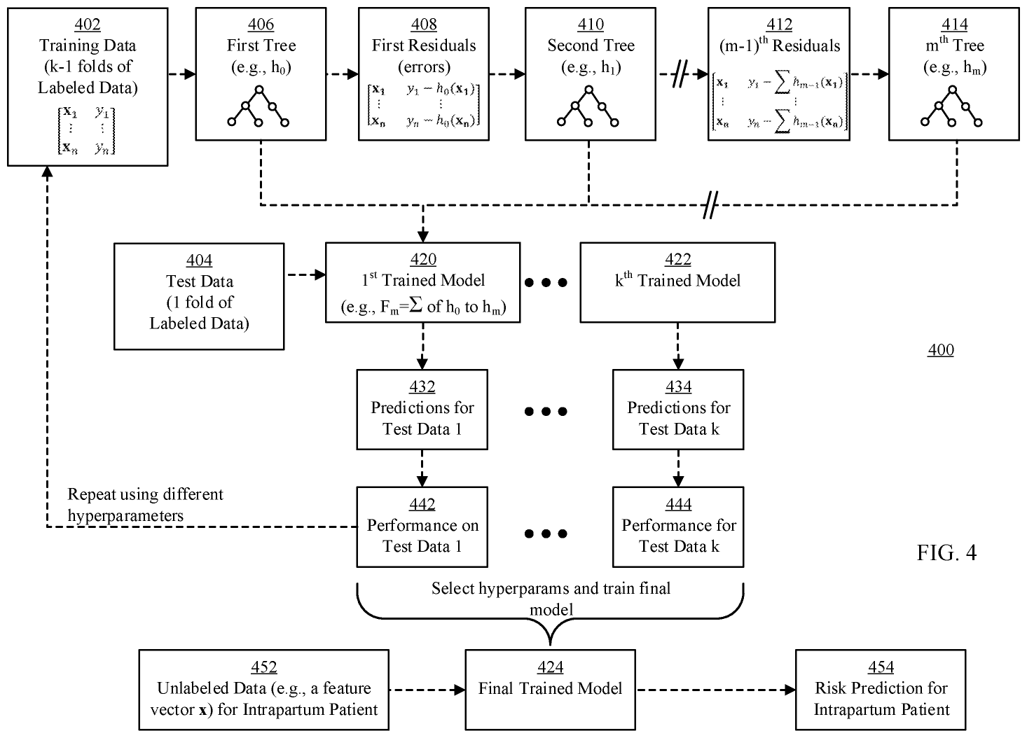

As described below in connection with FIG. 4, “Consortium on Safe Labor” database is a multicenter observational database that includes data associated with over 200,000 deliveries. In some embodiments, mechanisms described herein can use machine-learning techniques to generate a series of prediction models that incorporate both static and dynamic predictors, including patient baseline characteristics, most recent clinical assessment, and cumulative labor progress from admission. In some embodiments, the prediction models can be used to generate predictions that provide an alternative to current practices, which endorse the use of labor charts. In contrast to labor charts which set constant margins to safe labor course, prediction models trained using mechanisms described herein can make more individualized predictions that can promote clinical decisions that are more tailored to a particular patients circumstances using baseline and labor characteristics of the patient.

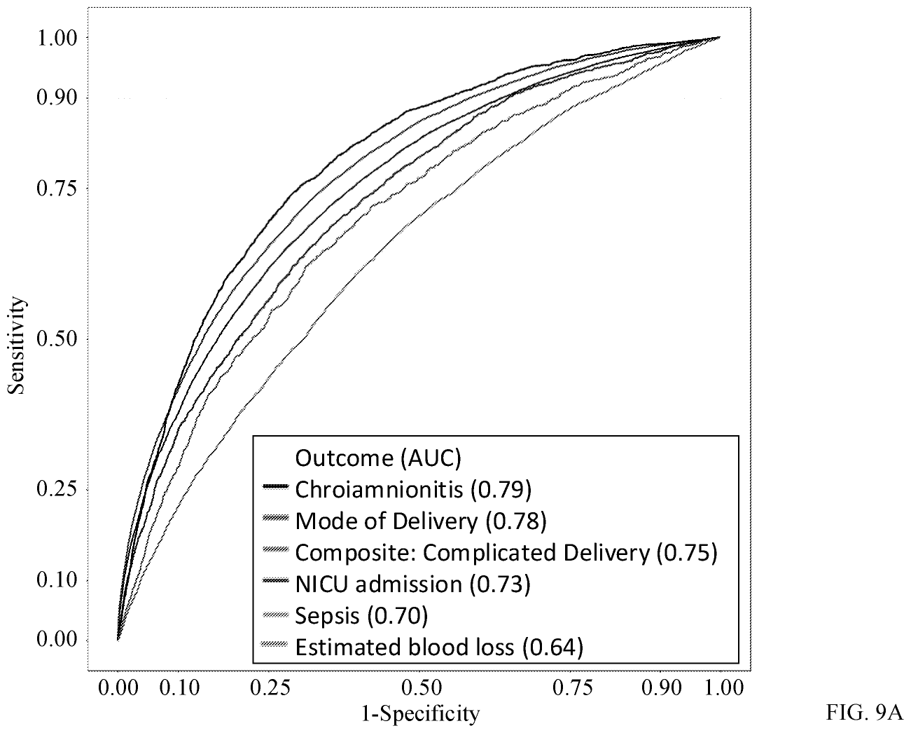

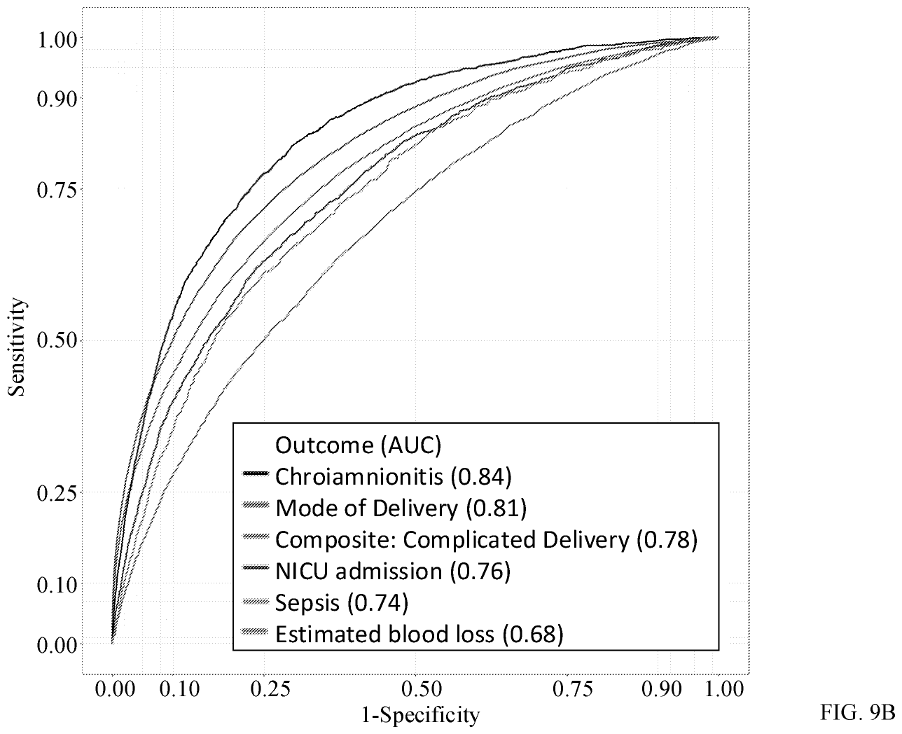

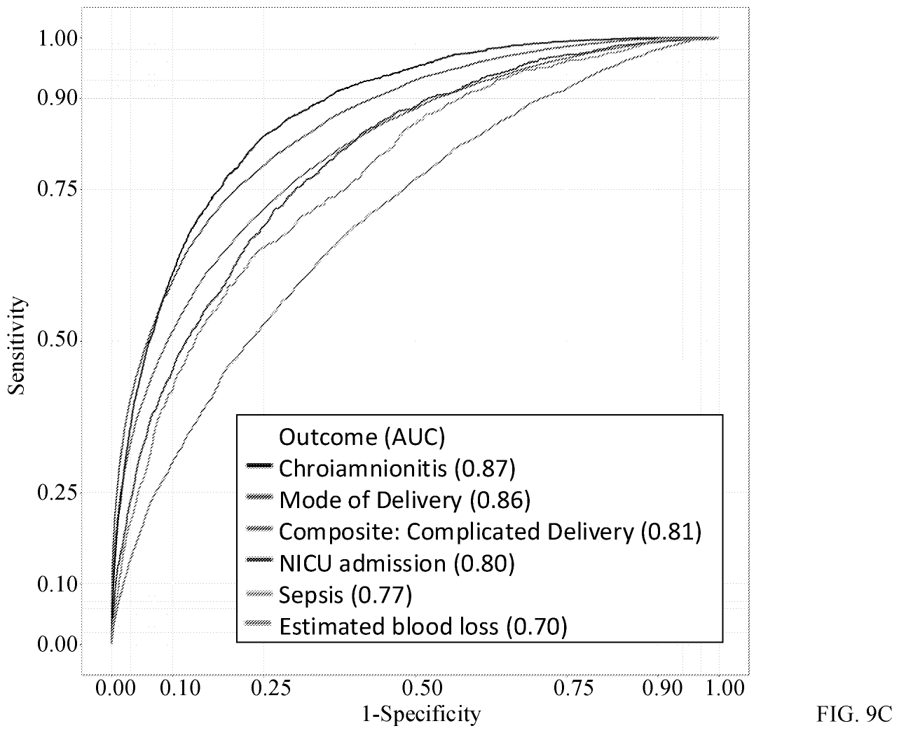

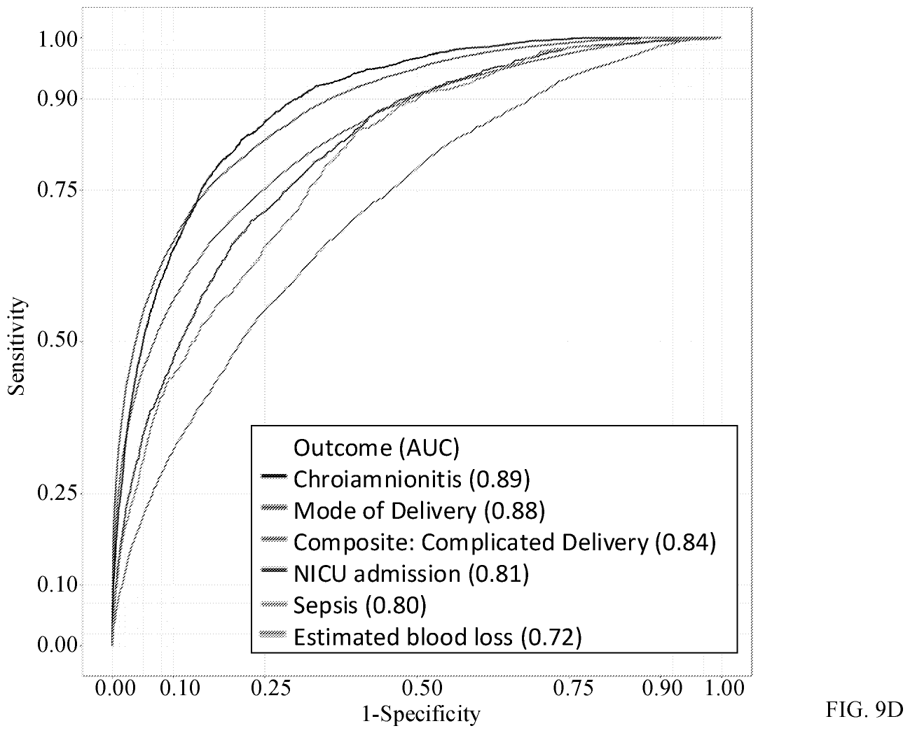

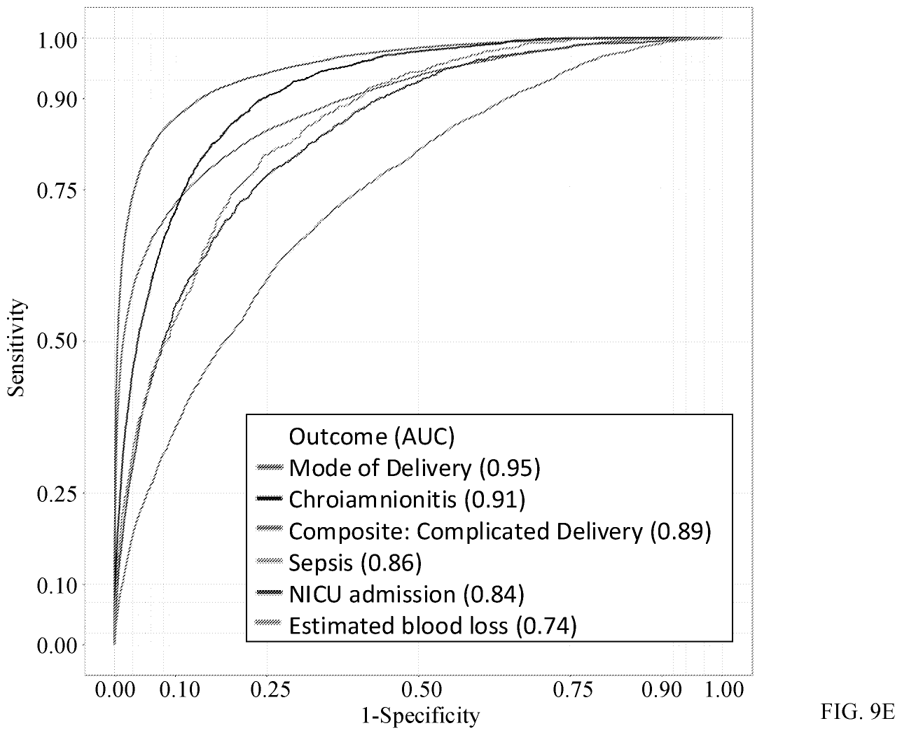

The challenges associated with creation of labor charts are not only attributed to the index population used to generate the data used to make the chart. Labor is a complex physiologic process and labor outcomes are likely to be influenced by several factors. These factors are either identifiable (e.g., can be determined at baseline), or unknown, yet indirectly reflected on labor course. Machine learning techniques can be useful to determine relationships between inputs and outcomes in large databases when the domain is poorly understood or when dynamic models are needed. Compared to conventional statistical methods, machine learning can minimize statistical assumptions, and can work by identifying patterns within data that are difficult or impossible to identify manually. Machine learning can also incorporate evolving risks during the labor progression in predicting outcomes of interest. As described below in connection with FIGS. 9A to 9E, the predictive power of models trained using mechanisms described herein is generally relatively strong. Unlike some conventional labor management techniques, mechanisms described herein do not explicitly rely on fixed definitions of latent labor, active labor, or rate of cervical dilation. In some embodiments, a graph of LRS output by one or more models in connection with progression of labor (e.g., as measured by cervical dilation) can be used to determine cumulative likelihood of safe labor taking into account the likelihood of cesarean delivery and/or adverse maternal and neonatal outcomes. For example, a patient's baseline LRS, LRS trend over time, and LRS graph in relation to one or more reference LRS graphs can promote intrapartum decision-making processes that may lead to a decreased incidence of adverse outcomes.

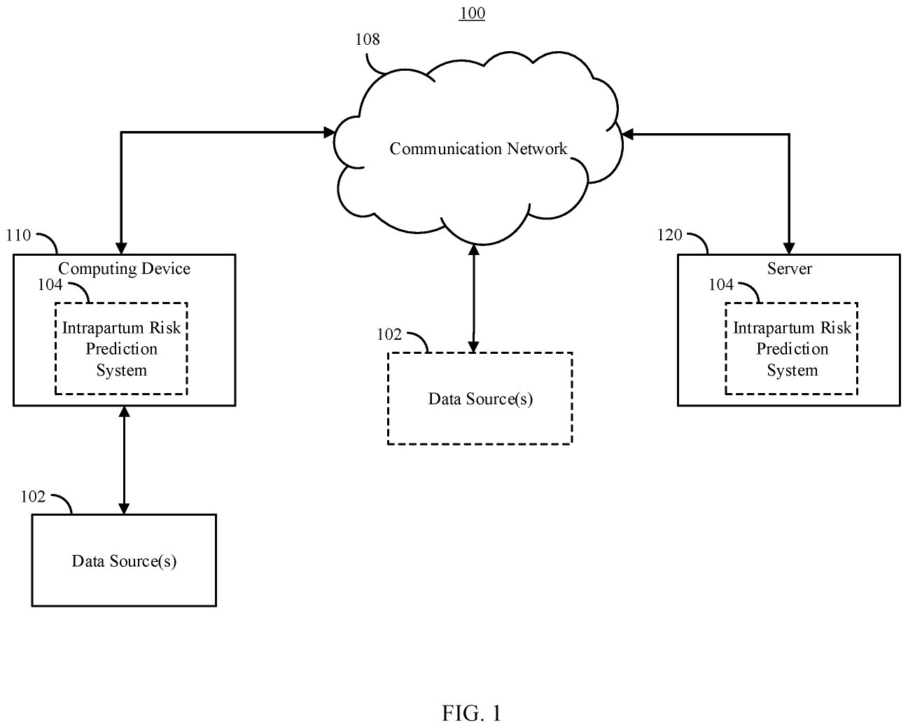

FIG. 1 shows an example of a system for intrapartum prediction of unfavorable labor outcomes in accordance with some embodiments of the disclosed subject matter. As shown in FIG. 1, a computing device 110 can receive variables related to the patient, variables related to the progression of labor, and/or other variables (e.g., environmental variables, variables related to the baby, etc.) from a data source 102 that stores such data, and/or from an input device. In some embodiments, computing device 110 can execute at least a portion of an intrapartum risk prediction system 104 to predict a risk that one or more unfavorable labor outcomes is likely to occur based on variables available at admission and/or up to the current point in labor.

Additionally or alternatively, in some embodiments, computing device 110 can communicate information about variables from data source 102 to a server 120 over a communication network 108 and/or server 120 can receive variables from data source 102 (e.g., directly and/or using communication network 108), which can execute at least a portion of intrapartum risk prediction system 104 to predict a risk that one or more unfavorable labor outcomes is likely to occur. In such embodiments, server 120 can return information to computing device 110 (and/or any other suitable computing device) indicative of a predicted risk of one or more unfavorable outcomes.

In some embodiments, computing device 110 and/or server 120 can be any suitable computing device or combination of devices, such as a desktop computer, a laptop computer, a smartphone, a tablet computer, a wearable computer, a server computer, a virtual machine being executed by a physical computing device, etc. As described below in connection with FIGS. 3-6, in some embodiments, computing device 110 and/or server 120 can receive labeled data (e.g., variables associated with labor and delivery) from one or more data sources (e.g., data source 102), and can format the variables for use in training a machine learning model to be used to provide intrapartum risk prediction system 104. In some embodiments, intrapartum risk prediction system 104 can use the labeled data to train a machine learning model(s) to predict risk of one or more unfavorable outcomes using unlabeled data from a patient that is in labor or expecting to go into labor.

In some embodiments, intrapartum risk prediction system 104 can receive unlabeled data (e.g., variables associated with the patient that is in labor or expected to go into labor) from one or more sources of data (e.g., data source 102), and can format the variables for input to the trained machine learning model(s). In some embodiments, intrapartum risk prediction system 104 can generate a predicted risk of one or more unfavorable outcomes based on available variables (e.g., LRS), and can present the results for a user (e.g., a physician, a midwife, a nurse, a paramedic, the patient, etc.).

In some embodiments, data source 102 can be any suitable source or sources of variables. For example, data source 102 can be an electronic medical records system. As another example, data source 102 can be a computing device used to collect data about the patient (e.g., via one or more devices, such as an SpO2 sensor, a blood pressure sensor, an end-tidal CO2 sensor, etc.). As yet another example, data source 102 can be an input device that facilitates manual data entry by a user. As still another example, data source 102 can be data stored in memory of computing device 110 and/or server 120 using any suitable format, such as using a database, a spreadsheet, a document with data entered using a comma separated value (CSV format), and/or any other suitable format.

In some embodiments, data source 102 can be local to computing device 110. For example, data source 102 can be incorporated with computing device 110 (e.g., using memory associated with computing device). As another example, data source 102 can be connected to computing device 110 by one or more cables, a direct wireless link, etc. Additionally or alternatively, in some embodiments, data source 102 can be located locally and/or remotely from computing device 110, and send data to computing device 110 (and/or server 120) via a communication network (e.g., communication network 108).

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, 5G NR, etc.), a wired network, etc. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc.

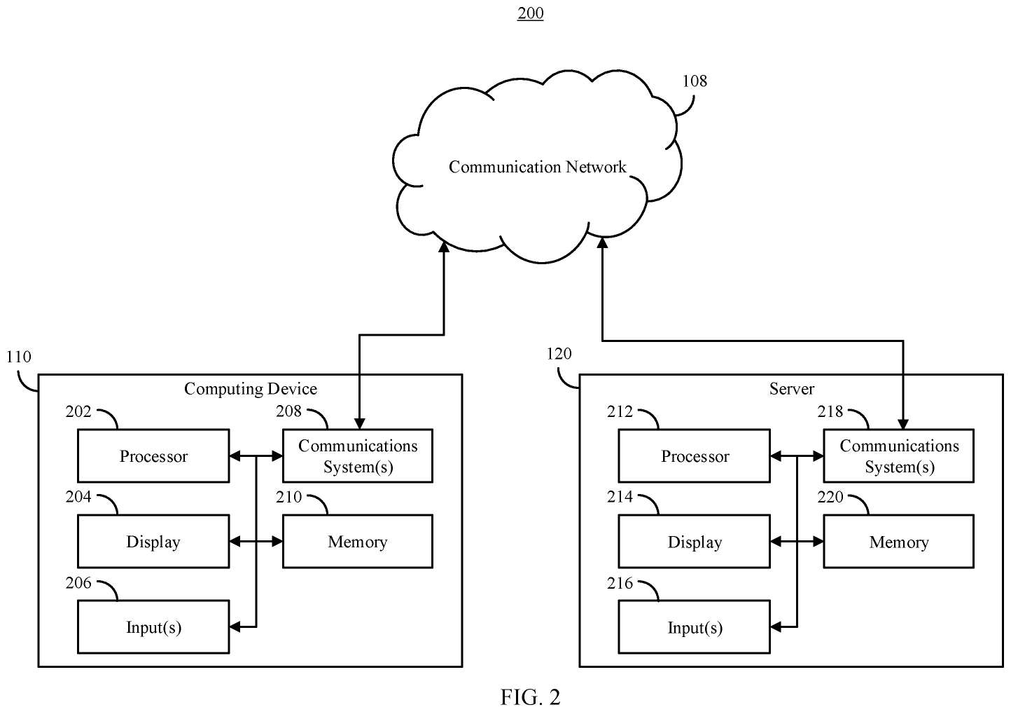

FIG. 2 shows an example 200 of hardware that can be used to implement computing device 110, and/or server 120 in accordance with some embodiments of the disclosed subject matter. As shown in FIG. 2, in some embodiments, computing device 110 can include a processor 202, a display 204, one or more inputs 206, one or more communication systems 208, and/or memory 210. In some embodiments, processor 202 can be any suitable hardware processor or combination of processors, such as a central processing unit (CPU), a graphics processing unit (GPU), a microcontroller (MCU), an application specification integrated circuit (ASIC), a field programmable gate array (FPGA), etc. In some embodiments, display 204 can include any suitable display devices, such as a computer monitor, a touchscreen, a television, etc. In some embodiments, inputs 206 can include any suitable input devices and/or sensors that can be used to receive user input, such as a keyboard, a mouse, a touchscreen, a microphone, etc.

In some embodiments, communications systems 208 can include any suitable hardware, firmware, and/or software for communicating information over communication network 108 and/or any other suitable communication networks. For example, communications systems 208 can include one or more transceivers, one or more communication chips and/or chip sets, etc. In a more particular example, communications systems 208 can include hardware, firmware and/or software that can be used to establish a Wi-Fi connection, a Bluetooth connection, a cellular connection, an Ethernet connection, etc.

In some embodiments, memory 210 can include any suitable storage device or devices that can be used to store instructions, values, etc., that can be used, for example, by processor 202 to present content using display 204, to communicate with server 120 via communications system(s) 208, etc. Memory 210 can include any suitable volatile memory, non-volatile memory, storage, or any suitable combination thereof. For example, memory 210 can include RAM, ROM, EEPROM, one or more flash drives, one or more hard disks, one or more solid state drives, one or more optical drives, etc. In some embodiments, memory 210 can have encoded thereon a computer program for controlling operation of computing device 110. In such embodiments, processor 202 can execute at least a portion of the computer program to present content (e.g., user interfaces, graphics, tables, reports, etc.), receive content from server 120, transmit information to server 120, etc.

In some embodiments, server 120 can include a processor 212, a display 214, one or more inputs 216, one or more communications systems 218, and/or memory 220. In some embodiments, processor 212 can be any suitable hardware processor or combination of processors, such as a CPU, a GPU, an MCU, an ASIC, an FPGA, etc. In some embodiments, display 214 can include any suitable display devices, such as a computer monitor, a touchscreen, a television, etc. In some embodiments, inputs 216 can include any suitable input devices and/or sensors that can be used to receive user input, such as a keyboard, a mouse, a touchscreen, a microphone, etc.

In some embodiments, communications systems 218 can include any suitable hardware, firmware, and/or software for communicating information over communication network 108 and/or any other suitable communication networks. For example, communications systems 218 can include one or more transceivers, one or more communication chips and/or chip sets, etc. In a more particular example, communications systems 218 can include hardware, firmware and/or software that can be used to establish a Wi-Fi connection, a Bluetooth connection, a cellular connection, an Ethernet connection, etc.

In some embodiments, memory 220 can include any suitable storage device or devices that can be used to store instructions, values, etc., that can be used, for example, by processor 212 to present content using display 214, to communicate with one or more computing devices 110, etc. Memory 220 can include any suitable volatile memory, non-volatile memory, storage, or any suitable combination thereof. For example, memory 220 can include RAM, ROM, EEPROM, one or more flash drives, one or more hard disks, one or more solid state drives, one or more optical drives, etc. In some embodiments, memory 220 can have encoded thereon a server program for controlling operation of server 120. In such embodiments, processor 212 can execute at least a portion of the server program to transmit information and/or content (e.g., a user interface, graphs, tables, reports, etc.) to one or more computing devices 110, receive information and/or content from one or more computing devices 110, receive instructions from one or more devices (e.g., a personal computer, a laptop computer, a tablet computer, a smartphone, etc.), etc.

FIG. 3 shows an example 300 of a system that can execute a group of models that can be used to predict intrapartum risk of unfavorable labor outcomes over time as dynamic variables change in accordance with some embodiments of the disclosed subject matter.

Over the course of the labor process, pelvic exam variables are repeatedly measured for each patient, and as such they are potentially correlated, which can present a major challenge for most machine learning models. In some embodiments, repeated observations for each patient prior to the current dilation can be aggregated to construct each of the intrapartum prediction models. For example, cross-sectional data for each intrapartum prediction model can be generated by aggregating the dynamic variables to three variables: the frequency (count) of the variable, the mean value of the variable, and the last observed value. In such an example, the dynamic variables can be aggregated using data points that were not used by a previous model, and can exclude data points (for dynamic variables) that were used as inputs (e.g., represented in aggregated inputs) to the previous model.

As shown in FIG. 3, in some embodiments, system 300 can use processor 202 to execute multiple trained machine learning models to predict the risk of one or more unfavorable outcomes (e.g., LRS, or one or more particular unfavorable outcomes such as CD) at various points during labor. In some embodiments, a baseline model 304 can be a machine learning model that has been trained to determine the risk that a patient will ultimately experience one or more unfavorable outcomes based on data available at the time of admission (e.g., when cervical dilation is unknown, or when cervical dilation has not yet reached a threshold dilation, such as 4 cm). In some embodiments, the baseline model can be based on static variables that do not change as labor progresses, such as demographic information of the patient, one or more physical characteristics associated with the patient (e.g., the number of times that the patient has carried a pregnancy to a viable gestational age sometimes referred to as parity, age, whether the patient has previously delivered via Cesarean, height, pre-pregnancy body mass index (BMI), gestational age, weight gain during pregnancy, whether the patient was experiencing contractions at the time of admission, etc.). In some embodiments, the static variables can be formatted as patient data at admission 302, which can be provided as input to baseline model 304, and baseline model 304 can provide an output indicative of a risk that the patient will experience one or more unfavorable outcomes based on patient data at admission 302. In some embodiments, the output from baseline model 304 can be output by processor 202 to memory 210 for presentation to a user and/or for use in determining risk at a more advanced stage of labor (e.g., as represented by the amount of cervical dilation).

In some embodiments, processor 202 can use an output by baseline model 304 to plot a risk at admission on a graph 350, and can use display 204 to render graph 350 for presentation to a user. In some embodiments, graph 350 can include information that can provide context to the user about the relative risk of the patient going on to have one or more unfavorable outcomes. For example, as shown in FIG. 3, lines can be plotted on graph 350 showing the average progression of risk scores for high risk patients (i.e., patients that did experience one or more unfavorable outcomes) and low risk patients (e.g., based on training data used to train the various models depicted in FIG. 3). A user (e.g., an obstetrician, a midwife, a nurse, etc.) can compare the plotted risk scores output from the models for the patient as labor has progressed to the average risk predictions for high risk and low risk patients. If the patient is tracking along the low risk line, the user can infer that the patient is at relatively low risk of experiencing one or more unfavorable outcomes, whereas if the patient is tracking along the high risk line, the user can infer that the patient is at relatively increased risk of experiencing one or more unfavorable outcomes. Additionally or alternatively, in some embodiments, the user can infer from the risk itself an absolute risk that the patient will experience one or more unfavorable outcomes. For example, if the risk output by one or more of the models is greater than 50%, the user can infer that the patient is more likely than not to experience one or more unfavorable outcomes. As described below, the models generally are more accurate as cervical dilation increases (e.g., as labor progresses), and the user can monitor the patient's risk or experiencing one or more unfavorable outcomes as labor progresses, and use the risk as information in a decision making process to determine whether to recommend CD to try to avoid unfavorable outcomes associated with a prolonged vaginal delivery or a continuation toward vaginal delivery to avoid unfavorable outcomes associated with CD.

In some embodiments, a 4 cm dilation model 314 can be a machine learning model that has been trained to determine the risk that a patient will ultimately experience one or more unfavorable outcomes based on data available up to 4 cm cervical dilation, and can be used when the patient has reached 4 cm dilation (e.g., which can be considered to be when the patient has entered into labor for the purposes of the models). In some embodiments, the baseline model can be based on static variables that do not change as labor progresses (e.g., variables used as inputs to baseline model 302), and dynamic variables that do change as labor progresses. Dynamic variables can include, for example, current cervical dilation, cervical effacement (e.g., categorized as 0-30%, 40-50%, 60-70%, 80% or more), head station (e.g., categorized as −3, −2, −1 or 0, +1 or +2), time interval elapsed between a current examination and the immediately preceding examination, change in cervical dilation between a current examination and the immediately preceding examination, fetal heart rate, and dilation delta (e.g., defined as a change in cervical dilation from the immediately preceding examination divided by the time interval between the two examinations), number of pelvic examinations conducted by medical practitioners (e.g., obstetricians, nurses, midwives, etc.), etc. In some embodiments, the static variables and dynamic variables can be formatted as patient data admission to 4 cm 312, which can be provided as input to 4 cm dilation model 314. In some embodiments, the output from baseline model 304 can also be used as an input to a 4 cm dilation model 314. In some embodiments, 4 cm dilation model 314 can provide an output indicative of a risk that the patient will experience one or more unfavorable outcomes based on patient data admission to 4 cm 312. In some embodiments, the output from 4 cm dilation model 314 can be output by processor 202 to memory 210 for presentation to a user and/or for use in determining risk at a more advanced stage of labor (e.g., as represented by the amount of cervical dilation). For example, the output from 4 cm dilation model 314 can be plotted on graph 350 and presented to a user for use in assessing the risk of the patient experiencing complications.

In some embodiments, as described above, dynamic variables can be aggregated prior to being provided as inputs to 4 cm dilation model 314. For example, information from various pelvic examinations can be aggregated by collecting data from each pelvic examination that occurred between admission and a final pelvic examination (e.g., at which dilation was first at or above 4 cm dilation), and one or more variables can be generated that represent the aggregation of the pelvic examinations. For example, the number of pelvic examinations can be used as an input. As another example, the average (e.g., mean, median) value of a variable (e.g., cervical dilation, cervical effacement, head station) can be used as an input. As yet another example, the value of a variable from the most recent examination (e.g., cervical dilation, cervical effacement, head station) can be used as an input.

In some embodiments, additional models (e.g., a 5 cm dilation model 324 that uses patient data admission to 5 cm 322, a 6 cm dilation model (not shown), and so on, up to a 10 cm dilation model 334 that uses patient data admission to 10 cm 332) can be machine learning models that have been trained to determine the risk that a patient will ultimately experience one or more unfavorable outcomes based on data available up to a current cervical dilation. In some embodiments, each successive model can use a risk score from a previous model as an input, and can use static data available at admission, and dynamic data associated with pelvic examinations (and/or other medical examinations) conducted during labor. In some embodiments, certain variables that were not associated with any particular cervical dilation in the training data, can be associated 10 cm dilation model 334, such as the presence of meconium in the amniotic fluid. Note that this is based on the limitations of the training data, and more finely labeled training data can be used to incorporate such variables into other models.

In some embodiments, when new data is received, which can include cervical dilation, a model corresponding to the current cervical dilation can be used to generate a new score. For example, cervical dilation checks are often performed at relatively regular internals during labor (e.g., about every two hours) and/or irregularly (e.g., if the patient exhibits changes in other variables that may indicate that labor is advancing or not advancing). In such an example, when a cervical check is performed, data generated during the cervical check can be provided to the processor 202 (e.g., via entry into an electronic medical record). If the cervical dilation associated with the newly provided data corresponds to a new model (e.g., a previous cervical dilation was 4 cm corresponding to 4 cm model 314, and the current cervical dilation is 5 cm corresponding to 5 cm model 324), processor 202 can use the provided data and any other data provided after the last cervical dilation check to generate aggregated dynamic variables. Otherwise, if the cervical dilation associated with the newly provided data corresponds to the same model (e.g., a previous cervical dilation was 4 cm corresponding to 4 cm model 314, and the current cervical dilation is <5 cm corresponding to 4 cm model 314), processor 202 can use the provided data and any other data provided after the last cervical dilation check to update the aggregated dynamic variables. In some embodiments, the newly received provided data can be used to generate a new risk score (e.g., if the cervical dilation corresponds to a new model), or an updated risk score (e.g., if the cervical dilation corresponds to the same model).

FIG. 4 shows an example of a flow for training and using mechanisms for intrapartum prediction of unfavorable labor outcomes in accordance with some embodiments of the disclosed subject matter. As shown in FIG. 4, labeled data can be used to train multiple machine learning models to predict a risk of a patient experiencing one or more unfavorable outcomes. In some embodiments, labeled data can include data sets for various patients for which data was collected at an appropriate point (or points) in time (e.g., before and during labor, and after delivery), and for which outcomes of the delivery are known. In some embodiments, the data associated with each patient can include various data points generated at various points in time, and which may or may not be associated with a particular cervical dilation. For example, the data associated with each patient can include one or more clinical variables (e.g., values indicative of age; sex; height; weight; diagnosis, such as diabetes, gestational diabetes, chronic hypertension, preeclampsia, etc.; parity; prior number of Cesarean deliveries; cervical dilation; cervical effacement; head station; etc.). As another example, the data associated with each patient can include non-clinical variables such as demographic information. As another example, the data associated with each patient can include ground truth information associated with the patient indicating whether one or more undesirable outcomes were associated with a particular labor and/or delivery.

In some embodiments, data from the DASH database can be used to generate labeled data used to train various machine learning models that can be used to predict intrapartum risk of unfavorable labor outcomes in accordance with some embodiments of the disclosed subject matter. The NICHD maintains the DASH database as a database that facilitates sharing of provider data that enables investigators to use de-identified data from NICHD funded research studies for the purpose of further research. A consortium of 12 clinical centers located in all 9 districts of the American College of Obstetricians and Gynecologists provided electronic obstetric, labor and newborn data between 2002 and 2008, which was used to create a large database, known as “Consortium on Safe Labor” database. As described above, this database was used by Zhang et al. to create contemporary labor curves. This database includes 228,438 deliveries with a total of 779 antepartum, intrapartum and postpartum variables. The de-identified version of this database was obtained with permission through DASH data use agreement, and was used to train multiple machine learning models to implement mechanisms for intrapartum prediction of unfavorable labor outcomes in accordance with some embodiments of the disclosed subject matter.

In some embodiments, data associated with patients falling into several categories can be removed from the data prior to generating labeled data for training and validation. For example, data associated with patients associated with one or more of the following were removed prior to generating training data: patients with multifetal pregnancy; patients that experienced intrauterine fetal death; patients that experienced preterm labor (defined as birth at less than 37 weeks of gestation); patients with fetal anomalies; patients that underwent elective CD (e.g., defined based on a failed induction; fetal malpresentation; cord prolapse; active herpetic lesion; CD performed prior to the onset of active labor, such as a CD performed at cervical dilation of 5 cm or less; and anyone with a history of CD, such as anyone having three or more prior CDs). Patients with inadequate documentation (e.g., defined as documentation of less than two cervical examinations), can also be excluded from the training data.

Out of 228,438 delivery episodes included in the “Consortium on Safe Labor” database, 66,586 episodes were eligible for use as training data based on the criteria described above. Within the 66,586 episodes, mean maternal age at admission was 26.95±6.48 years, mean parity was 0.92±1.23, and pre-pregnancy BMI was 25.24±5.58 kg/m², with a mean weight gain during pregnancy of 14.71±5.92 kg. Race and ethnicity were diverse; 21,155 (31.8%) were identified as White, 23,128 (34.7%) were identified as African-American, 14,862 (22.3%) were identified as Hispanic, 2,745 (4.1%) were identified as Asian/Pacific Islander, 193 (0.3%) were identified as multi-racial, 2,072 (3.1%) were identified as belonging to another race(s), and 2,431 (3.7%) were reported as unknown. The mean gestational age at admission to labor was 39.35±1.13 weeks of gestation. Medical complications of pregnancy included 10,305 (2.0%) patients that were diagnosed with pregestational diabetes during that pregnancy, 1,041 (1.6%) that were diagnosed with gestational diabetes, 1,106 (1.7%) had gestational hypertension, 1,085 (1.6%) had preeclampsia, and 1,085 (1.6%) had chronic hypertension. The rate of prior CD was 2,394 (3.6%) for the entire cohort. Delivery was initiated by labor induction in 31,932 (48.0%) of the episodes. Detailed demographic and clinical characteristics of the population represented in the training and validation data are included in TABLE 1.

TABLE 1

| | Patients with | | Patients with | | | | |
|---|---|---|---|---|---|---|---|
| | favorable outcomes | | unfavorable outcomes | | All patients | | |
| Variables* | (N = 52,147) | | (N = 14,439) | | (N = 66,586) | | p value |
| Maternal age in years | 26.80 ± 6.40 | | 27.47 ± 6.73 | | 26.95 ± 6.48 | | <0.001 |
| Parity | 1.03 ± 1.26 | | 0.52 ± 1.01 | | 0.92 ± 1.23 | | <0.001 |
| History of macrosomia in previous pregnancies | 850 | (1.6) | 115 | (0.8) | 965 | (1.4) | <0.001 |
| Prepregnancy BMI (kg/m²) | 25.05 ± 5.42 | | 25.94 ± 6.05 | | 25.24 ± 5.58 | | <0.001 |
| Pregestational diabetes | 941 | (1.8) | 364 | (2.5) | 1305 | (2.0) | <0.001 |
| History of heart disease | 473 | (0.9) | 127 | (0.9) | 600 | (0.9) | 0.757 |
| Antenatal positive GBS status | 10852 | (20.8) | 3103 | (21.5) | 13955 | (21.0) | 0.076 |
| Smoking | 2674 | (5.1) | 651 | (4.5) | 3325 | (5.0) | 0.003 |
| Cerclage placement in current pregnancy | 111 | (0.2) | 28 | (0.2) | 139 | (0.2) | 0.659 |
| Gestational hypertension | 796 | (1.5) | 310 | (2.1) | 1106 | (1.7) | <0.001 |
| Preeclampsia | 711 | (1.4) | 374 | (2.6) | 1085 | (1.6) | <0.001 |
| Eclampsia | 31 | (0.1) | 9 | (0.1) | 40 | (0.1) | 0.900 |
| Superimposed preeclampsia | 364 | (0.7) | 212 | (1.5) | 576 | (0.9) | <0.001 |
| Chronic hypertension | 549 | (1.1) | 229 | (1.6) | 778 | (1.2) | <0.001 |
| Gestational diabetes | 725 | (1.4) | 316 | (2.2) | 1041 | (1.6) | <0.001 |
| Intrauterine growth restriction | 292 | (0.6) | 79 | (0.5) | 371 | (0.6) | 0.855 |
| Oligohydramnios | 967 | (1.9) | 413 | (2.9) | 1380 | (2.1) | <0.001 |
| Polyhydramnios | 74 | (0.1) | 43 | (0.3) | 117 | (0.2) | <0.001 |
| Maternal weight on admission in kg | 81.43 ± 16.29 | | 84.00 ± 17.79 | | 81.99 ± 16.66 | | <0.001 |
| Gestational age on admission | 39.31 ± 1.11 | | 39.50 ± 1.17 | | 39.35 ± 1.13 | | <0.001 |
| Maternal ethnicity | | | | | | | <0.001 |
| White | 16807 | (32.2) | 4348 | (30.1) | 21155 | (31.8) | |
| Black | 18055 | (34.6) | 5073 | (35.1) | 23128 | (34.7) | |
| Hispanic | 11707 | (22.4) | 3155 | (21.9) | 14862 | (22.3) | |
| Asian/Pacific Islander | 2054 | (3.9) | 691 | (4.8) | 2745 | (4.1) | |
| Multi-racial | 153 | (0.3) | 40 | (0.3) | 193 | (0.3) | |
| Others | 1567 | (3.0) | 505 | (3.5) | 2072 | (3.1) | |
| Unknown | 1804 | (3.5) | 627 | (4.3) | 2431 | (3.7) | |
| Maternal height in meters | 1.63 ± 0.07 | | 1.62 ± 0.07 | | 1.63 ± 0.07 | | <0.001 |
| Alcohol use | 1134 | (2.2) | 291 | (2.0) | 1425 | (2.1) | 0.242 |
| Weight change during pregnancy in kgs | 14.47 ± 5.82 | | 15.58 ± 6.20 | | 14.71 ± 5.92 | | <0.001 |
| ECV in this pregnancy | 92 | (0.2) | 16 | (0.1) | 108 | (0.2) | 0.083 |
| Pre-pregnancy weight in kgs | 66.95 ± 15.68 | | 68.39 ± 17.27 | | 67.26 ± 16.05 | | <0.001 |
| Fetal sex | | | | | | | <0.001 |
| Female | 26164 | (50.2) | 6568 | (45.5) | 32732 | (49.2) | |
| Male | 25932 | (49.7) | 7836 | (54.3) | 33768 | (50.7) | |
| Ambiguous | 1 | (0.0) | 1 | (0.0) | 2 | (0.0) | |
| Unknown | 50 | (0.1) | 34 | (0.2) | 84 | (0.1) | |
| Previous CDs | | | | | | | <0.001 |
| 0 | 50683 | (97.2) | 13509 | (93.6) | 64192 | (96.4) | |
| 1 | 1420 | (2.7) | 833 | (5.8) | 2253 | (3.4) | |
| 2 | 39 | (0.1) | 87 | (0.6) | 126 | (0.2) | |
| Induction of labor | 23586 | (45.2) | 8346 | (57.8) | 31932 | (48.0) | <0.001 |
| Meconium stained amniotic fluid | | | | | | | <0.001 |
| No | 47375 | (90.8%) | 12422 | (86.0%) | 59797 | (89.8%) | |
| Yes (unspecified) | 4639 | (8.9%) | 1954 | (13.5%) | 6593 | (9.9%) | |
| Thin | 81 | (0.2%) | 34 | (0.2%) | 115 | (0.2%) | |
| Moderate | 1 | (0.0%) | 2 | (0.0%) | 3 | (0.0%) | |
| Thick | 51 | (0.1%) | 27 | (0.2%) | 78 | (0.1%) | |
| Method of labor induction | | | | | | | |
| AROM | 1292 | (2.5) | 268 | (1.9) | 1560 | (2.3) | <0.001 |
| Prostaglandin E1 | 1067 | (2.0) | 719 | (5.0) | 1786 | (2.7) | <0.001 |
| Mechanical methods | 43 | (0.1) | 41 | (0.3) | 84 | (0.1) | <0.001 |
| Prostaglandin E2 | 412 | (0.8) | 148 | (1.0) | 560 | (0.8) | 0.006 |
| Oxytocin | 12427 | (23.8) | 3952 | (27.4) | 16379 | (24.6) | <0.001 |
| Method of ROM | | | | | | | <0.001 |
| AROM | 30380 | (58.3%) | 8275 | (57.3%) | 38655 | (58.1%) | |
| SROM | 20012 | (38.4%) | 5713 | (39.6%) | 25725 | (38.6%) | |
| PROM | 14 | (0.0%) | 8 | (0.1%) | 22 | (0.0%) | |
| Others | 356 | (0.7%) | 46 | (0.3%) | 402 | (0.6%) | |
| Unknown | 1385 | (2.7%) | 397 | (2.7%) | 1782 | (2.7%) | |

As described above, unfavorable outcomes can include various outcomes associated with labor and/or delivery, and can be any of the following: unsuccessful vaginal delivery (e.g., leading to CD in active labor), postpartum hemorrhage (e.g., defined as estimated blood loss that is greater than 1000 ml) or need for transfusion of blood products, suspected or confirmed intra-amniotic infection (IAI), shoulder dystocia, neonatal admission to intensive care unit (NICU), an APGAR score below 7 at 5 minutes, an umbilical arterial pH below 7.00, neonatal hypoxemic ischemic encephalopathy (HIE), neonatal ventilation use or continuous positive airway pressure (CPAP) therapy, neonatal intracranial hemorrhage (ICH), neonatal sepsis, or neonatal death. In some embodiments, the mechanisms described herein can generate an LRS that is indicative of a probability of an unfavorable labor outcome which can be defined as a composite of the preceding unfavorable outcomes. In the episodes that were eligible for use as training data based on the criteria described above unfavorable labor outcomes were reported in 14,439 (21.68%) of total delivery episodes included in the training and validation data. Of these, 10,466 (15.7%) deliveries were intrapartum CDs, 2,395 (3.6%) were diagnosed with TAT, 1,261 (2.0%) had postpartum hemorrhage, and 3,743 (5.6%) of delivered neonates were admitted to NICU. The incidence of neonatal sepsis and neonatal death were 880 (1.3%) and 49 (0.1%), respectively. In some embodiments, missing values can be imputed for a particular episode (e.g., with not more than 30% missing observations) using any suitable technique or combination of techniques. For example, in some embodiments, missing values can be imputed using random forest imputation techniques, such as missForest techniques described in Stekhoven, et al., “MissForest—non-parametric missing value imputation for mixed-type data.” Bioinformatics (2011), which is hereby incorporated by reference herein in its entirety.

In some embodiments, each model (e.g., baseline model 304, 4 cm dilation model 314, 5 cm dilation model 324, etc.) can be a gradient boosting machine (GBM)-based model, which can be trained using any suitable technique or combination of techniques. For example, GBMs can generally achieve the best results by tuning various hyperparameters for optimal performance along one or more dimensions (e.g., to maximize sensitivity, to maximize specificity, to maximize area under the receiver operating characteristic curve (AUC), etc.). In a particular example, a grid search can be performed by repeatedly training GBMs from the same set of training data with different hyperparameters, and the highest performing hyperparameters can be selected for use in training a final model using all of the training data. In a more particular example, a grid for each combination of hyperparameters can be established, and the best combination can be selected using a 10-fold cross-validation training technique in which the training data is randomly partitioned into 10 mutually exclusive subsets (or folds). Training of the GBM can be performed using 9 folds, and the hold-out fold can be used for testing the performance of the trained model. The entire procedure can be repeated until each fold is used as the test fold, and the performance can be averaged across all test folds with confidence intervals computed.

In some embodiments, data associated with each patient can be formatted as a vector x with a length corresponding to the total number of features on which the machine learning model is to be trained, and a value y representing the diagnosis associated with the patient. For example, if the patient data to be used in training includes n variables, the vector x can have a length of n with each position corresponding to a particular variable and having a value indicative of the value of the variable. In some embodiments, the outcome for each patient can be coded as a binary value (e.g., 0 indicating no unfavorable outcomes, or 1 indicating at least one unfavorable outcome). As another example, the outcome for each patient can be coded as a factor having multiple levels, with an integer value corresponding to a particular unfavorable outcome. In a more particular example, no unfavorable outcomes, intrapartum CD, TAT, postpartum hemorrhage, admission to NICU, and neonatal sepsis/death can be coded as integer values 0 to 5, with the presence of a higher coded outcome taking precedence over a lower coded outcome. As yet another example, the outcome for each patient can be coded as a vector with each element representing a particular unfavorable outcome with a 1 indicating the presence of the outcome and a 0 indicating the absence of the outcome. Note that these are merely examples, and outcomes can be coded using other schemes.

In some embodiments, the training data can be grouped into any suitable number of folds that each have a distribution of outcomes that is similar to the overall distribution of outcomes. In some embodiments, a set of training data 402 can include all but one of the folds. In general, cross-validation is an approach to training statistical learning models that provides a way of assessing how a model can be expected to generalize to different datasets. For example, if the labeled data has been divided into ten folds, training data 402 can include nine of the ten folds to be used to train a first machine learning model. In such embodiments, a fold (or folds) not included in training data 402 can be used as test data 404, which can be used to evaluate the performance of a trained model. As described above, in a ten-fold cross-validation, the training data can be divided into ten relatively equal sections which can be referred to as folds, each of which maintains roughly the same class balance of the whole training dataset. A model can be trained on nine of the ten folds and can be assessed using the tenth fold. This can be repeated ten times using a different assessment fold each time, and the performance of the models on each fold can be compared and/or aggregated. Note that ten-fold cross-validation is merely an example, and any suitable number of folds (i.e., k-folds) can be used.

In some embodiments, a grid search can be conducted to determine values for various hyperparameters, such as maximum number of trees (m), learning rate (η), shrinkage, and maximum interaction depth. In such embodiments, multiple models can be generated using various combinations of hyperparameter values, and can be evaluated to determine which hyperparameters generate superior performing models. After evaluating the performance of the various models and selecting hyperparameters that produce best results, a final model can be produced by training on all available labeled data.

In some embodiments, training data 402 can be used to generate a first tree 406 using any suitable technique or combination of techniques. For example, first tree 406 can be a simple tree that is generated using training data 402 and one or more hyperparameters, such as a maximum interaction depth that can limit the number of splits (e.g., if-then statements) allowed between the root and the deepest leaf node, that are allowed in each of the constituent trees. In some embodiments, first tree 406 can be automatically generated using any suitable tree generation technique or combination of techniques. For example, first tree 406 can be generated by determining at each node which feature of the remaining features that have not been selected in the current tree can be used to split the patients associated with that node into new nodes that minimize prediction error. This can be done recursively until a stopping condition is reached, such as a minimum number of patients (e.g., one, two, etc.) has been reached, a maximum depth has been reached, or if another division would fail to improve prediction accuracy (e.g., if the current group is homogenous in class, dividing the group again may not provide additional predictive power). In a more particular example, if training data 402 includes 6,659 patients, those 6,659 patients can be associated with a root node, and can be divided by determining a feature (e.g., a variable, such as parity) along which to split the group. If a feature is categorical (e.g., prior C-section, baby sex: male, polyhydramnios: no, etc.), the group can be divided based on category membership, whereas if a feature is continuous, the feature can be discretized prior to building the tree and/or model (e.g., age can be discretized into multiple binary features, e.g., <20, <26, >30, etc.), and a single discretized feature can be used to split the group associated with the root node. While a single tree could provide some predictive power, decision trees are considered weak learners and alone provide limited accuracy, performance is typically heavily biased by the data that the decision tree is trained on. Note that in some embodiments, an initial tree (e.g., first tree 406) can be a decision tree that is trained using the actual outcome data. However, a first tree can also be generated using a constant that minimizes error (i.e., the observed outcomes y used for training can be set to the same value for each patient, such as no unfavorable outcome, which is closest to an average outcome).

In some embodiments, the accuracy of a final trained model can be increased using any suitable technique or combination of techniques. For example, GBM techniques can be used to increase the predictive power of first tree 406 by iteratively adding additional trees that each reduce the error when added to all of the previous trees. In such embodiments, the predictions made by the first tree 406 for each patient can be used to generate a first set of residuals 408 that represent the error in the prediction. In some embodiments, the error can be generated using any suitable loss function, which can be used to generate pseudo-residual values and first residuals 408 can be the pseudo-residuals.

In some embodiments, first residuals 408 can then be used to train a second tree 410, which can be used to generate second residuals, and so on, until a set of (m−1)^{th} residuals 412 are used to train a final m^{th} tree 414. In some embodiments, the number of trees m used to generate a final model is a hyperparameter that can be set at a particular number or determined based on whether generating an additional tree (e.g., an additional decision tree) would improve the performance of the overall model.

In some embodiments, a trained model 420 can be an aggregation of all of the individual trees 406, 410, . . . , 414, and a trained model can be generated for each unique combination of folds (e.g., models 1-k can be generated with a k^{th} model 422 generated based on the k^{th} set of labeled data). In some embodiments, test data 404 that was reserved from each combination of training data can be used to evaluate the performance of each of the trained models (e.g., first trained model 420 can be evaluated based on the fold reserved from training data 402, while k^{th} model 422 can be evaluated based on the fold reserved from k^{th} training data). In some embodiments, first trained model 420 generates a set of predictions 432 using the test data 404, k^{th} model 422 generates a set of predictions 434 using the k^{th} test data, and each other model is used to make a similar set of predictions based on corresponding test data that was not used during the training process.

In some embodiments, the performance of each model can be calculated based on a comparison of the predictions (e.g., predictions 432 to 434) to the labels associated with the corresponding test data (e.g., based on test data 404, etc.), to generate performance metrics 442 to 444 corresponding to each of the k models. Additionally, in some embodiments, each combination of training data and test data can be used to generate multiple models with various hyperparameters in a grid search operation. For example, the same combination of training data (e.g., training data 402) and test data (e.g., test data 404) can be used to generate multiple different trained models 420 to 422 using different combinations of hyperparameters (e.g., a first set of models 420 to 422 using a first combination of hyperparameters, a second set of models 420 to 422 using a second combination of hyperparameters, and so on). In a more particular example, for each set of hyperparameters in the search space that is selected, a k-fold cross validation process can be used to determine performance characteristics associated with the set of hyperparameters. A set of hyperparameters that has the most desirable performance characteristics can be used to train the final model. In some embodiments, the search space can include any suitable range of maximum interactions depth, learning rate (sometimes referred to as shrinkage), and number of trees.

In some embodiments, a final trained model 424 can be generated using hyperparameters that generated the best performance (e.g., where best can be determined using various different metrics). For example, after determining a set of hyperparameters that generate a desired performance, a new GBM of decision trees can be generated using all of the data (i.e., all k folds of data, rather than k−1 folds for training with one fold withheld for testing) and the final set of hyperparameters.

Alternatively, in some embodiments, final trained model 424 can be based on one or more of the trained models (e.g., models 420 to 422). For example, in some embodiments, the model that minimized one or more undesirable metrics (e.g., false negatives, false positives, etc.) or maximized one or more desirable metrics (e.g., specificity, true positives, true negatives, etc.) can be selected as a best performing model and used as final trained model 424. As another example, the performance of each of the k models can be evaluated, and the models can be combined to generate final model 424. In a more particular example, each trained model 420 to 422 can be assigned a weight based on the performance associated with that model (e.g., performance 442 to 444 respectively), and a final output of final trained model 424 can be based on a weighted combination of each of the k trained models.

In some embodiments, after training is complete, unlabeled data 452 corresponding to a patient that is currently in labor or expected to be going into labor can be provided as input to final trained model 424, and final trained model 424 can provide a prediction 454 that is indicative of the risk that the patient will experience one or more unfavorable outcomes based on the input data.

In some embodiments, the flow described above in connection with FIG. 4 can be used to train multiple prediction models used to predict risk at various points during labor. For example, a baseline model (e.g., baseline model 304) can be trained first using the flow described above in connection with FIG. 4, and an output of the baseline model can be used as part of the training data when training a subsequent model (e.g., 4 cm dilation model 314).

In some embodiments, the data used from the database to train machine learning models for predicting risk at different points during labor can be different. For example, data used to train the baseline model can be selected from only the data that would have been available at admission, while data used to train a later model (e.g., 4 cm dilation model 314) can be selected from data that would have been available through 4 cm cervical dilation (e.g., prior to 5 cm dilation), and data used to train later models can be selected from data that would have been associated with the corresponding cervical dilation (e.g., data associated with cervical dilations of at least 5 cm but less than 6 cm can be used to train 5 cm dilation model 324, data associated with cervical dilations of at least 6 cm but less than 7 cm can be used to train a 6 cm dilation model, data associated with cervical dilation of at least 10 cm can be used to train 10 cm dilation model 334, etc.).

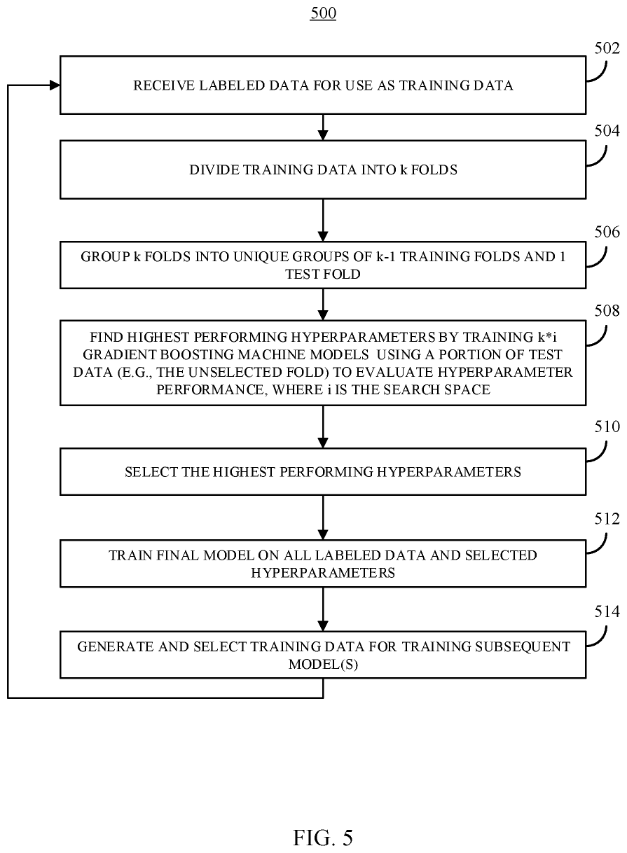

FIG. 5 shows an example 500 of a process for training a machine learning model to predict intrapartum risks of unfavorable labor outcomes in accordance with some embodiments of the disclosed subject matter. As shown in FIG. 5, at 502, process 500 can receive labeled data for use as training data. As described above, process 500 can receive the labeled data from any suitable source, and the training data can include data related to any suitable variables, such as clinical variables and/or demographic variables.

At 504, process 500 can divide the labeled data into k folds that each have a similar distribution of outcomes to the overall distribution. In some embodiments, any suitable technique or combination of techniques can be used to divide the labeled training data, such as by randomly assigning patients with each outcome across the k folds.

At 506, process 500 can generate groupings of the folds into unique combinations of k−1 folds as training data and 1 fold as validation and/or testing data, such that each fold is used as a test fold with the k other folds as training folds.

At 508, process 500 can find a set of highest performing hyperparameters by training k*i decision tree-based GBMs, each having different hyperparameters, where i is a search space of the hyperparameters. As described above in connection with FIG. 4, the performance of each model can be measured during and/or after training to determine which hyperparameters produce the highest performing models. For example, accuracy, positive predictive value, negative predictive value, and other suitable performance characteristics can be calculated for one or more thresholds. In a more particular example, such performance characteristics can be calculated for naïve thresholds (e.g., over 50%).

In some embodiments, process 500 can perform a search over any suitable hyperparameters such as the maximum number of trees (m) allowed, the maximum interaction depth allowed, and learning rate. The number of trees can be used to limit the total number of decision trees included in the model. The interaction depth can be used to limit the number of splits that are allowed in each of the constituent trees, which can control the degree of interactions between predictor variables. For example, an interaction depth of one implies a model that is purely additive, while an interaction depth of two allows for first order interactions. More generally, an interaction depth of n allows interactions up to order n−1. The shrinkage hyperparameter can be used to modify the learning rate of the algorithm as each additional tree is added to the model. As described above, using grid search techniques to select hyperparameters can include trained and evaluated models identically across a wide selection of parameter combinations. Such techniques are generally more computationally intensive than other techniques such as random search or Bayesian optimization, but can account for a greater variety of parameters. However, such other techniques can also be used in lieu of grid search techniques.

While mechanisms described herein are generally described in connection with a multinomial target distribution, binomial target distributions can also be used. For example, multiple models can be built which can include models that each make a prediction of whether a particular unfavorable outcome is likely to occur, such as whether intrapartum CD is likely, whether IAI is likely to occur, etc. In such an example, the outputs of the different models can be used in connection with one another to predict a composite likelihood that any unfavorable outcome is likely to occur, and/or a likelihood that a particular unfavorable outcome is likely to occur.

At 510, process 500 can select the best performing hyperparameters based on the performance of the models trained at 508 on test data. In some embodiments, performance can be evaluated using any suitable technique or combination of techniques, such as by comparing the area under the receiver operating characteristic curve (AUC) for models that make a binomial (two-class) prediction. The performance can be evaluated based on the predictions made for the out-of-sample cross-validation results. In some embodiments, the hyperparameters for the final model can be selected based on the model that.

At 512, process 500 can train a final model using all of the labeled data and the hyperparameters selected at 510. For example, process 500 can train a decision tree-based GBM with a multinomial classifier using the hyperparameters selected at 510. Other than using all of the data (e.g., not withholding a test set), training of the final model can be performed using techniques described above for training models used to evaluate various hyperparameters.

At 514, process 500 can generate and/or select training data to train a next model in a series of models that can be used to model the risk that one or more unfavorable labor outcomes will occur as labor develops in later stages. For example, risk scores can be generated using the model trained at 512 to be used in training a subsequent model that uses the risk score from an earlier model as an input. Additionally, in some embodiments, at 514, data from the intervening period between the end of the period modeled by the model trained at 512 and the subsequent model can be aggregated. After generating and/or selecting training data for training the subsequent model(s) at 514, process 500 can return to 502 and repeat process 500 for the subsequent model until each of the final models desired to model labor (e.g., through 10 cm cervical dilation) are trained at 512.

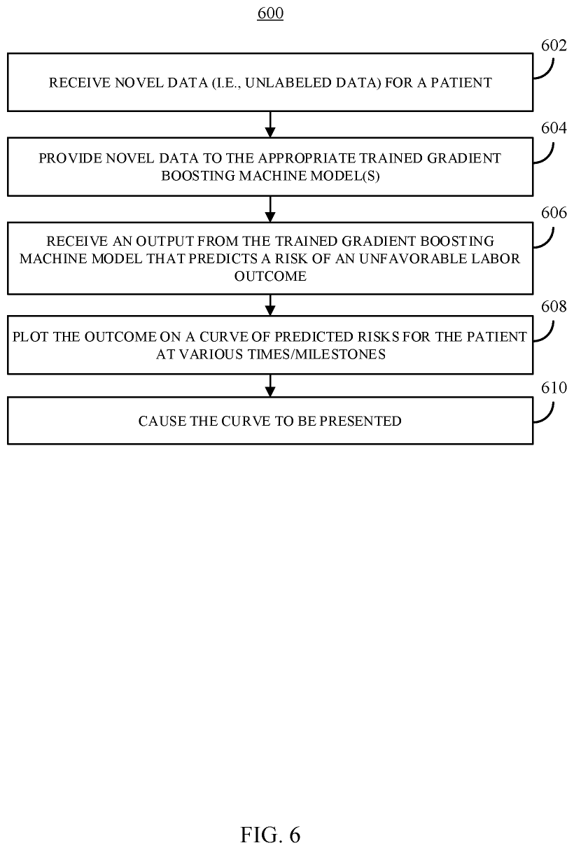

FIG. 6 shows an example 600 of a process for using a machine learning model to predict intrapartum risk of unfavorable labor outcomes in accordance with some embodiments of the disclosed subject matter. As shown in FIG. 6, process 600 can begin at 602 by receiving novel data associated with a patient that is in labor or is expected to go into labor. For example, process 600 can receive variables associated with the patient from any suitable source (e.g., data source 102, a user interface being executed by a computing device executing process 600).

At 604, process 600 can provide novel data to a trained GBM model in a format that matches a format of the training data. For example, process 500 can provide the novel data to baseline model 304, 4 cm dilation model 314, etc., a final GBM model trained at 512, and/or final trained model 424.

At 606, process 600 can receive an output from the trained GBM model that is a prediction of the intrapartum risk that the patient will experience one or more unfavorable labor outcomes. In some embodiments, the output can be in any suitable format. For example, the output can be in a format that provides a risk score as a percentage likelihood that the patient will experience one or more unfavorable labor outcomes.

At 608, process 600 can plot the outcome on a curve of predicted risks for the patient at various times and/or milestones. For example, process 600 can plot the outcome on a curve of predicted risk as a function of cervical dilation. In some embodiments, for example as described above in connection with FIG. 3, process 600 can plot the outcome in connection examples showing average risks for patients that did and did not experience the one or more unfavorable outcomes (or at least one of the unfavorable outcomes). Additionally or alternatively, at 608, process 600 can generate a report using the novel data and the predicted risk. In some embodiments, process 600 can generate updated data for the same model. For example, the same model (e.g., 5 cm dilation model 324) can produce multiple risk values at different times corresponding to multiple cervical checks that produce data corresponding to cervical dilations in the range corresponding to the model. In such an example, process 600 can plot the updated data using any suitable technique or combination of techniques. In a particular example, process 600 can use the updated value to replace the previous value. As another more, particular example, process 600 can use the updated value and any previous values to generate a range to illustrate variance in the risk scores, and can plot the updated value and the range.

At 610, process 600 can cause the curve of predicted risks to be presented to a user in connection with any suitable contextual information that can be used by the user in a decision making process to determine whether to recommend that the patient undergo an intrapartum CD or other procedure to attempt to ameliorate the risks involved. For example, process 600 can cause the curve and any suitable contextual information to be presented to a physician or other medical professional (e.g., a nurse, a midwife) treating the patient (e.g., using computing device 110) in response to a request from the physician or other medical professional and/or in response to the physician accessing an electronic medical record associated with the patient. Additionally or alternatively, in some embodiments, process 600 can cause the report to be presented to a user.

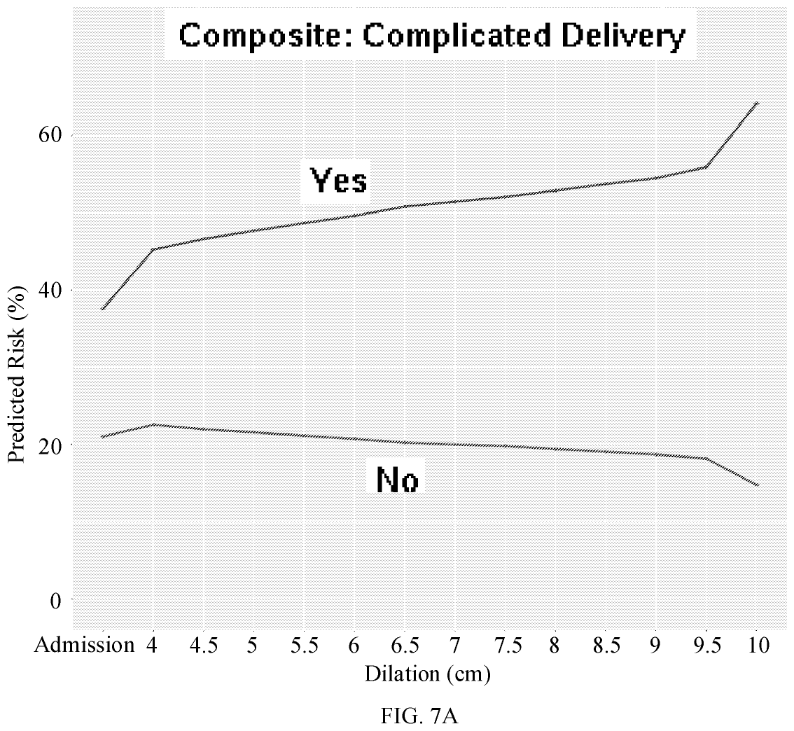

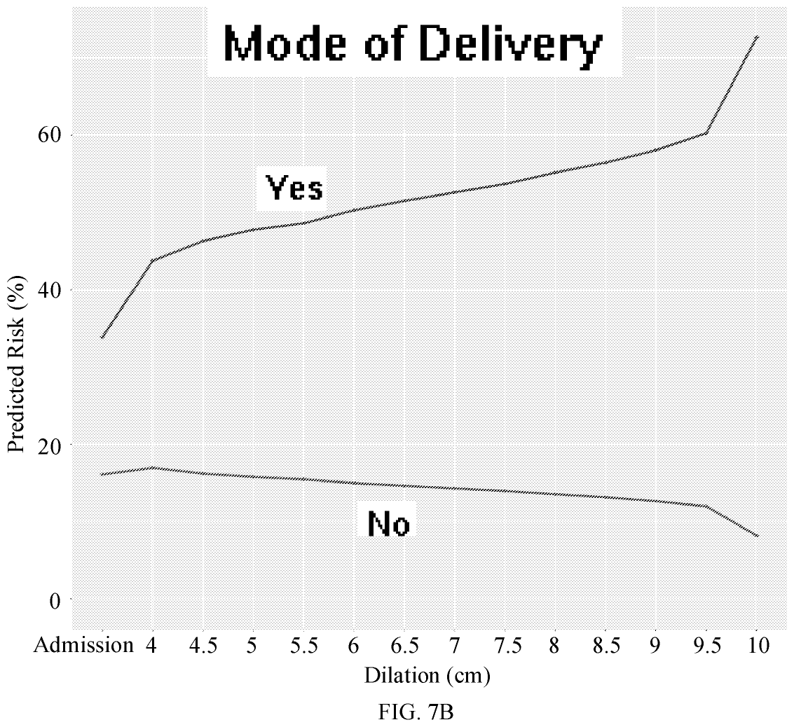

FIG. 7A shows an example of trends in average risk scores over labor progress for deliveries that were complicated (“YES”) and not complicated (“NO”) predicted by machine learning models implemented in accordance with some embodiments of the disclosed subject matter. As shown in FIG. 7A, LRS values were generated for patients in the “Consortium on Safe Labor” database, averaged, and plotted against cervical dilation. The LRS values were generated by a series of models that were trained in accordance with some embodiments of the disclosed subject matter (e.g., as described above in connection with FIGS. 4 and 5), and used a risk score output from a previous model in the series as an input to a current model. FIG. 7A demonstrates that the LRS trend among patients that had favorable versus unfavorable composite outcomes is relatively consistent.

As shown in FIG. 7A, patients with unfavorable composite outcomes had a baseline LRS score above 35%. Their scores at 4 to 6 cm were between 45% and 50% and consistently trended up beyond 60% over increasing cervical dilation. By contrast, baseline LRS was below 25% among patients with favorable composite outcomes, and their scores trended down from 23% at 4 cm, to 20% at 7 cm, and finally to 15% at 10 cm.

FIG. 7B shows an example of trends in average risk scores over labor progress for intrapartum Cesarean delivery (“YES”) and vaginal deliveries (“NO”) predicted by machine learning models implemented in accordance with some embodiments of the disclosed subject matter. Similar to what is shown in FIG. 7A, risk of failed vaginal delivery trended up from 34% on admission to 72% at 10 cm in women delivered by intrapartum CD. In women who had successful vaginal delivery, the risk of failed vaginal delivery was below 20% and trended below 10% at 10 cm (FIG. 3).

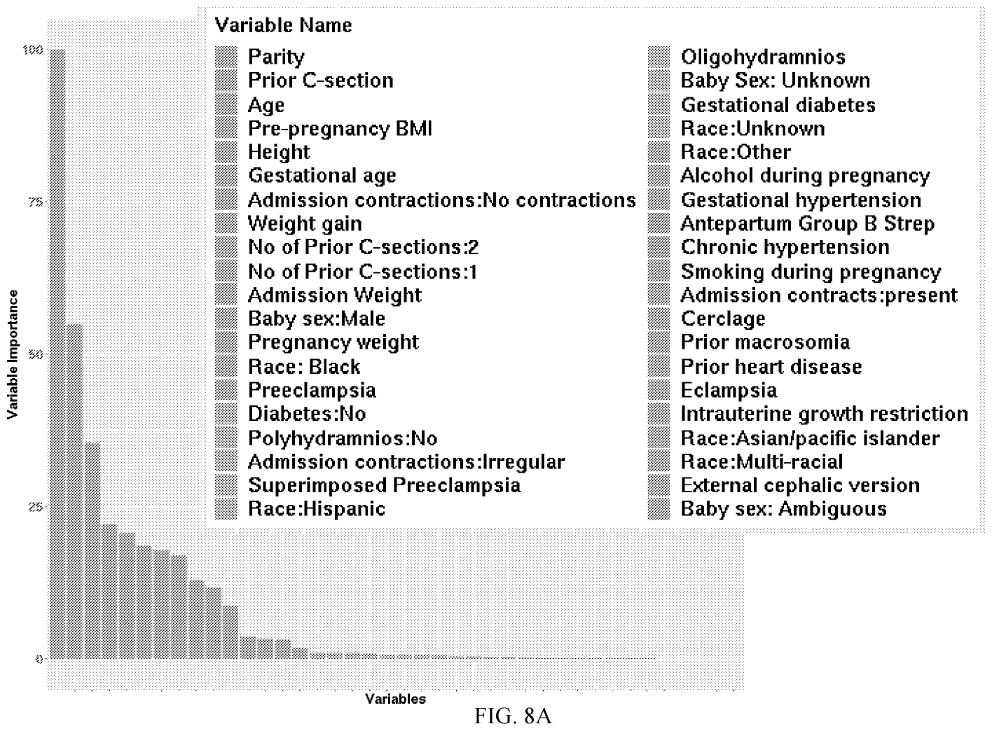

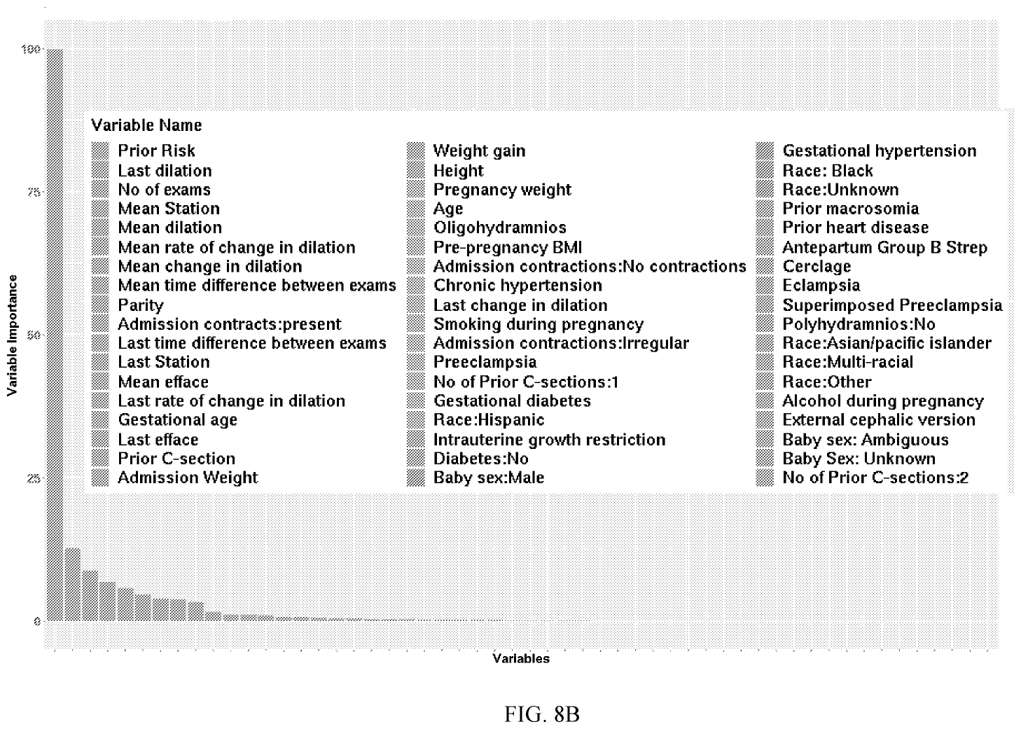

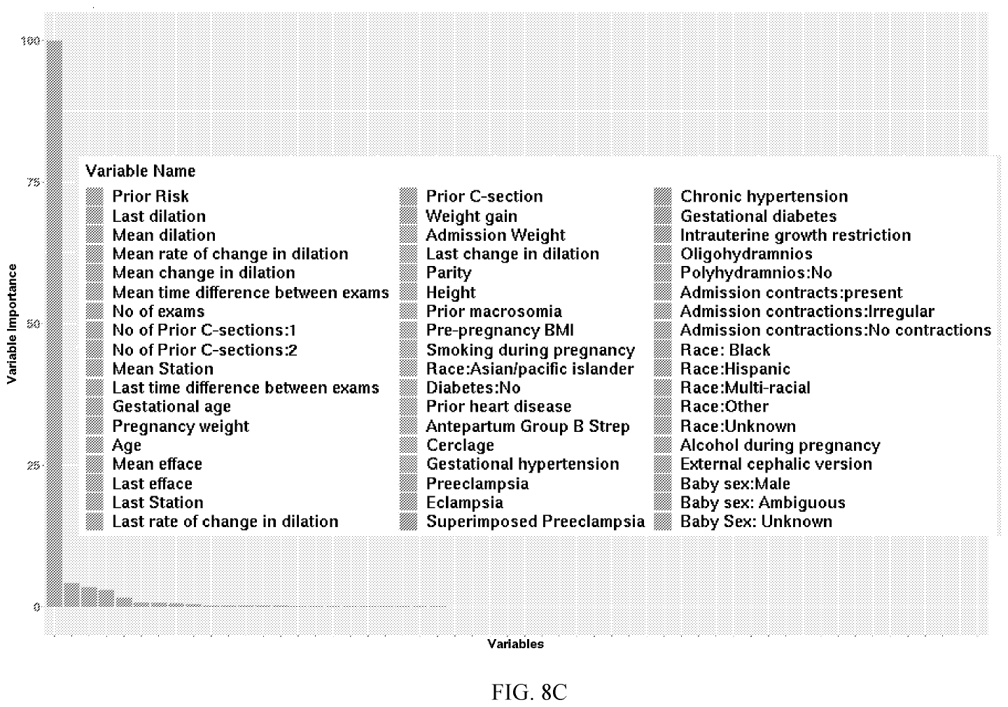

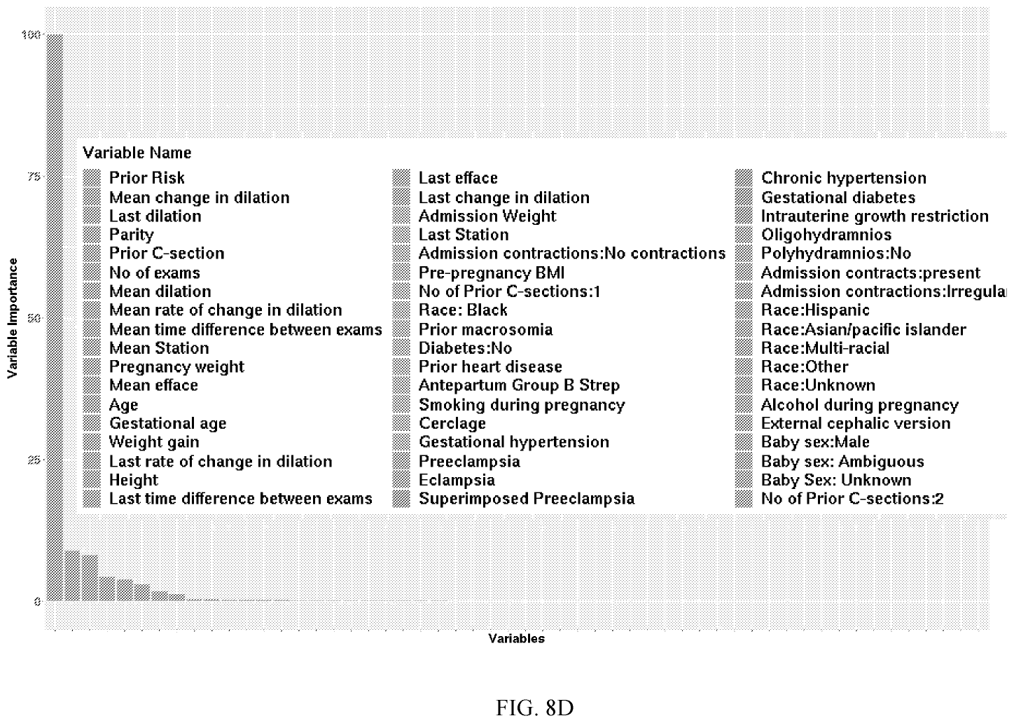

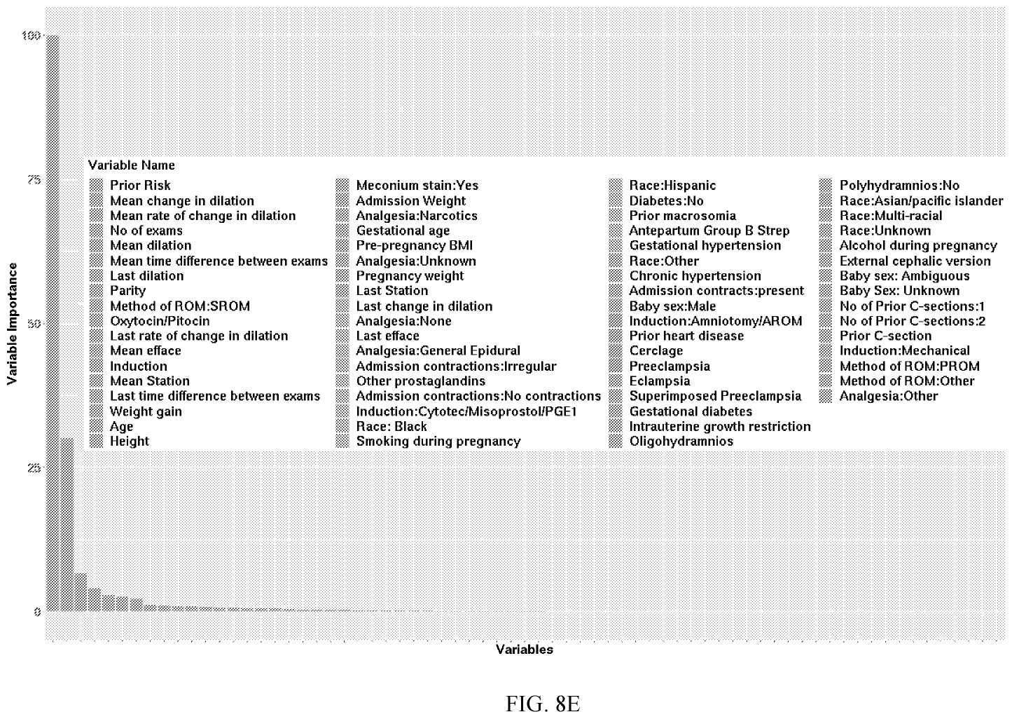

FIGS. 8A to 8E show examples of variables that had the largest impact on the predicted risk score for machine learning models implemented in accordance with some embodiments of the disclosed subject matter to predict a risk of a composite of unfavorable labor outcomes based on variables measurable at admission, and based on variables measurable up to 4 cm, 6 cm, 8 cm, and 10 cm cervical dilation, respectively.

On admission, a machine learning-based prediction model implemented in accordance with some embodiments of the mechanisms described herein performed at sensitivity of 0.69 (95% confidence interval (CI) 0.68-0.70) and specificity of 0.68 (95% CI 0.67-0.69) in predicting unfavorable labor outcome; AUC was 0.75 (95% CI 0.75-0.75). TABLE 2 includes performance metrics for a series of models for a composite of unfavorable labor outcomes, and for intrapartum Cesarean delivery in isolation. As shown in FIG. 8A, the most contributing independent variable to the baseline model was parity (i.e., the number of pregnancies carried to a viable gestational age). Other significant variables included prior Cesarean delivery, maternal age, maternal pre-pregnancy BMI, height, gestational age at admission, absence of uterine contractions on admission, and maternal weight gain during pregnancy. As shown in TABLE 2, the diagnostic performance of intrapartum prediction models trended up with advancement of cervical dilation; model sensitivity increased gradually from 0.70 (95% CI 0.69-0.70) at 4 cm to 0.79 (95% CI 0.78-0.80) at 10 cm. Similarly, model specificity rose from 0.72 (95% CI 0.71-0.73) at 4 cm to 0.84 (95% CI 0.83-0.85) at 10 cm. As shown in FIGS. 8B to 8E, the most significant variable for all intrapartum models was prior risk score from the previous model. Other contributing factors to these models included cervical dilation at last examination, number of cervical examinations, current head station, cervical dilation change, current cervical dilation and dilation delta. The spectrum of contributing factors and the magnitude of their contribution to baseline and intrapartum prediction models are shown in FIGS. 8A to 8E.

TABLE 2

| | Cervical | | | | | |
|---|---|---|---|---|---|---|
| | dilation | | | | | |
| Outcome | (in cm) | Error | AUC | Sensitivity | Specificity | PPV |
| Composite | Baseline | 0.31 | 0.75 | 0.69 | 0.68 | 0.42 |
| outcome | | (0.31, 0.32) | (0.75, 0.75) | (0.68, 0.70) | (0.67, 0.69) | (0.42, 0.42) |
| (unfavorable | 4 | 0.29 | 0.78 | 0.70 | 0.72 | 0.50 |
| labor | | (0.29, 0.30) | (0.77, 0.78) | (0.69, 0.70) | (0.71, 0.73) | (0.49, 0.51) |
| outcomes) | 5 | 0.28 | 0.80 | 0.70 | 0.74 | 0.52 |
| | | (0.28, 0.28) | (0.80, 0.80) | (0.70, 0.71) | (0.73, 0.75) | (0.52, 0.53) |
| | 6 | 0.27 | 0.81 | 0.72 | 0.75 | 0.55 |
| | | (0.26, 0.27) | (0.81, 0.81) | (0.70, 0.73) | (0.74, 0.77) | (0.54, 0.55) |
| | 7 | 0.25 | 0.83 | 0.73 | 0.76 | 0.56 |
| | | (0.25, 0.26) | (0.82, 0.83) | (0.72, 0.74) | (0.75, 0.77) | (0.55, 0.57) |
| | 8 | 0.25 | 0.84 | 0.75 | 0.75 | 0.56 |
| | | (0.24, 0.25) | (0.83, 0.84) | (0.74, 0.76) | (0.74, 0.77) | (0.54, 0.57) |
| | 9 | 0.24 | 0.85 | 0.76 | 0.76 | 0.57 |
| | | (0.24, 0.24) | (0.84, 0.85) | (0.75, 0.77) | (0.76, 0.77) | (0.56, 0.57) |
| | 10 | 0.19 | 0.89 | 0.79 | 0.84 | 0.67 |
| | | (0.18, 0.19) | (0.89, 0.90) | (0.78, 0.80) | (0.83, 0.85) | (0.66, 0.68) |
| Intrapartum | Baseline | 0.29 | 0.78 | 0.71 | 0.70 | 0.37 |
| Cesarean | | (0.29, 0.30) | (0.77, 0.78) | (0.70, 0.72) | (0.69, 0.71) | (0.36, 0.37) |
| delivery | 4 | 0.27 | 0.81 | 0.72 | 0.74 | 0.46 |
| | | (0.26, 0.27) | (0.81, 0.82) | (0.71, 0.74) | (0.73, 0.75) | (0.45, 0.47) |
| | 5 | 0.24 | 0.84 | 0.75 | 0.76 | 0.49 |
| | | (0.24, 0.24) | (0.84, 0.84) | (0.75, 0.76) | (0.76, 0.77) | (0.48, 0.49) |
| | 6 | 0.23 | 0.86 | 0.76 | 0.79 | 0.52 |
| | | (0.22, 0.23) | (0.85, 0.86) | (0.74, 0.77) | (0.78, 0.79) | (0.51, 0.53) |
| | 7 | 0.21 | 0.87 | 0.78 | 0.79 | 0.53 |
| | | (0.21, 0.22) | (0.87, 0.88) | (0.78, 0.79) | (0.78, 0.80) | (0.52, 0.54) |
| | 8 | 0.20 | 0.88 | 0.78 | 0.82 | 0.56 |
| | | (0.20, 0.20) | (0.88, 0.89) | (0.77, 0.79) | (0.81, 0.83) | (0.55, 0.57) |
| | 9 | 0.19 | 0.90 | 0.80 | 0.83 | 0.58 |
| | | (0.18, 0.19) | (0.90, 0.90) | (0.79, 0.80) | (0.82, 0.83) | (0.57, 0.59) |
| | 10 | 0.12 | 0.95 | 0.87 | 0.90 | 0.72 |
| | | (0.11, 0.12) | (0.95, 0.95) | (0.86, 0.88) | (0.89, 0.91) | (0.71, 0.74) |

FIGS. 9A to 9E show examples of performance of a machine learning model implemented in accordance with some embodiments of the disclosed subject matter to predict a risk of a composite of unfavorable labor outcomes based on variables measurable at admission, and based on variables measurable up to 4 cm, 6 cm, 8 cm, and 10 cm cervical dilation, respectively. As shown in FIGS. 9A to 9E, AUC of intrapartum prediction models at admission, 4 cm, 6 cm, 8 cm, and 10 cm show a similar trend of increasing performance for each of the various unfavorable outcomes that were measured. For example, in the case of composite complicated delivery, AUC shows a trend of increasing performance (0.75 at admission, 0.78 “95% CI 0.77-0.78” at 4 cm, 0.89 “95% CI 0.89-0.90” at 10 cm).

### Further Examples Having a Variety of Features

Example 1: A method for predicting a risk of one or more unfavorable labor outcomes in a patient, the method comprising: generating a feature vector that includes a first plurality of values and a second plurality of values, wherein the first plurality of values corresponds to a respective plurality of static variables that are knowable at a time a patient goes into labor, and the second plurality of values corresponds to a respective plurality of dynamic variables that are associated with a particular time during labor, the second plurality of values includes at least a most recent cervical dilation value; providing the feature vector to a trained machine learning model, wherein the trained machine learning model was trained using a plurality of labeled feature vectors associated with a respective plurality of patients associated with one or more known labor outcomes, wherein each of the plurality of labeled feature vectors included values corresponding to the plurality of static variables and the plurality of dynamic variables associated with a respective patient and associated with a cervical dilation value in a range that includes the most recent cervical dilation value, and each of the plurality of labeled feature vectors is associated with an indication of one or more unfavorable outcomes experienced by the respective patient; receiving, from the trained machine learning model, an output indicative of a risk that the patient will experience at least one of the one or more unfavorable outcomes; and causing information indicative of the risk to be presented to a user to aid the user in determining whether to recommend intrapartum Cesarean delivery for the patient.

Example 2: The method of Example 1, wherein the trained machine learning model is a gradient boosting machine model comprising a plurality of decision trees.

Example 3: The method of any one of Examples 1 or 2, further comprising: receiving, from a baseline machine learning model, a baseline output indicative of a risk that the patient will experience at least one of the one or more unfavorable outcomes based on variables knowable at the time the patient went into labor, wherein the baseline machine learning model was trained using a second plurality of labeled feature vectors associated with a respective plurality of patients associated with one or more known labor outcomes, wherein each of the second plurality of labeled feature vectors included values corresponding to the plurality of static variables associated with a respective patient and omitted any dynamic variables associated with the respective patient, and each of the plurality of labeled feature vectors is associated with an indication of one or more unfavorable outcomes experienced by the respective patient; and wherein generating the feature vector comprises including the baseline output in the feature vector.

Example 4: The method of any one of Examples 1 to 3, wherein the trained machine learning model is trained to predict risk that the patient will experience at least one of the one or more unfavorable outcomes based on data collected through 4 centimeters (cm) cervical dilation, and wherein the most recent cervical dilation value is a cervical dilation value that is at least 4 cm cervical dilation and less than 5 cm.

Example 5: The method of any one of Examples 1 to 4, wherein the plurality of static variables includes variables corresponding to parity, a binary indication of whether the patient has previously delivered via Cesarean, and the patient's age.

Example 6: The method of any one of Examples 1 to 5, wherein the plurality of dynamic variables includes variables corresponding to cervical dilation, cervical effacement, and head station.

Example 7: The method of any one of Examples 1 to 6, further comprising: plotting the outcome on a graph, wherein the graph includes a curve representing average risk scores for patients that did not experience unfavorable outcomes and a second curve representing risk scores for patients that experienced one or more unfavorable outcomes; and causing the graph to be presented as the information indicative of the risk.

Example 8: The method of Example 7, further comprising: generating a second feature vector that includes the first plurality of values and a third plurality of values, wherein the third plurality of values corresponds to a respective plurality of dynamic variables that are associated with a second particular time during labor, including at least a most recent cervical dilation value that exceeds an upper limit of the range associated with the trained model; providing the second feature vector to a second trained machine learning model, wherein the second machine learning model was trained using a second plurality of labeled feature vectors associated with the respective plurality of patients associated with the one or more known labor outcomes, wherein each of the second plurality of labeled feature vectors included values corresponding to the plurality of static variables and the plurality of dynamic variables associated with a respective patient and associated with a cervical dilation value in a second range that includes the most recent cervical dilation value included in the third plurality of values, and each of the second plurality of labeled feature vectors is associated with an indication of one or more unfavorable outcomes experienced by the respective patient; receiving, from the second trained machine learning model, a second output indicative of an updated risk that the patient will experience at least one of the one or more unfavorable outcomes; generating an updated graph by plotting the second outcome on the graph; and causing the updated graph to be presented to the user to aid the user in determining whether to recommend intrapartum Cesarean delivery for the patient.

Example 9: The method of Example 8, wherein the range associated with the trained machine learning model includes cervical dilation from about 4 cm to less than 5 cm and the second range associated with the second trained machine learning model includes cervical dilation from about 5 cm to less than 6 cm.

Example 10: A system comprising: at least one hardware processor that is configured to: perform a method of any one of Examples 1 to 9.

Example 11: A non-transitory computer readable medium containing computer executable instructions that, when executed by a processor, cause the processor to perform a method of any one of Examples 1 to 9.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above described steps of the processes of FIGS. 5 and 6 can be executed or performed in any order or sequence not limited to the order and sequence shown and described in the figures. Also, some of the above steps of the processes of FIGS. 5 and 6 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

1. A system for predicting a risk of one or more unfavorable labor outcomes in a patient, the system comprising:
at least one hardware processor that is programmed to:
generate a feature vector that includes a first plurality of values and a second plurality of values,
wherein the first plurality of values corresponds to a respective plurality of static variables that are knowable at a time a patient goes into labor, and
the second plurality of values corresponds to a respective plurality of dynamic variables that are associated with a particular time during labor, the second plurality of values includes at least a most recent cervical dilation value;
provide the feature vector to a trained machine learning model, wherein the trained machine learning model was trained using a plurality of labeled feature vectors associated with a respective plurality of patients associated with one or more known labor outcomes,
wherein each of the plurality of labeled feature vectors included values corresponding to the plurality of static variables and the plurality of dynamic variables associated with a respective patient and associated with a cervical dilation value in a range that includes the most recent cervical dilation value, and
each of the plurality of labeled feature vectors is associated with an indication of one or more unfavorable outcomes experienced by the respective patient;
receive, from the trained machine learning model, an output indicative of a risk that the patient will experience at least one of the one or more unfavorable outcomes; and
cause information indicative of the risk to be presented to a user to aid the user in determining whether to recommend intrapartum Cesarean delivery for the patient.

2. The system of claim 1, wherein the trained machine learning model is a gradient boosting machine model comprising a plurality of decision trees.

3. The system of claim 2, wherein the at least one hardware processor is further programmed to:
receive, from a baseline machine learning model, a baseline output indicative of a risk that the patient will experience at least one of the one or more unfavorable outcomes based on variables knowable at the time the patient went into labor, wherein the baseline machine learning model was trained using a second plurality of labeled feature vectors associated with a respective plurality of patients associated with one or more known labor outcomes,
wherein each of the second plurality of labeled feature vectors included values corresponding to the plurality of static variables associated with a respective patient and omitted any dynamic variables associated with the respective patient, and
each of the plurality of labeled feature vectors is associated with an indication of one or more unfavorable outcomes experienced by the respective patient; and
include the baseline output in the feature vector.

4. The system of claim 3, wherein the trained machine learning model is trained to predict risk that the patient will experience at least one of the one or more unfavorable outcomes based on data collected through 4 centimeters (cm) cervical dilation, and
wherein the most recent cervical dilation value is a cervical dilation value that is at least 4 cm cervical dilation and less than 5 cm.

5. The system of claim 1, wherein the plurality of static variables includes variables corresponding to parity, a binary indication of whether the patient has previously delivered via Cesarean, and the patient's age.

6. The system of claim 1, wherein the plurality of dynamic variables includes variables corresponding to cervical dilation, cervical effacement, and head station.

7. The system of claim 1, wherein the at least one hardware processor is further programmed to:
plot the outcome on a graph, wherein the graph includes a curve representing average risk scores for patients that did not experience unfavorable outcomes and a second curve representing risk scores for patients that experienced one or more unfavorable outcomes; and
cause the graph to be presented as the information indicative of the risk.

8. The system of claim 7, wherein the at least one hardware processor is further programmed to:
generate a second feature vector that includes the first plurality of values and a third plurality of values,
wherein the third plurality of values corresponds to a respective plurality of dynamic variables that are associated with a second particular time during labor, including at least a most recent cervical dilation value that exceeds an upper limit of the range associated with the trained model;
provide the second feature vector to a second trained machine learning model, wherein the second machine learning model was trained using a second plurality of labeled feature vectors associated with the respective plurality of patients associated with the one or more known labor outcomes,
wherein each of the second plurality of labeled feature vectors included values corresponding to the plurality of static variables and the plurality of dynamic variables associated with a respective patient and associated with a cervical dilation value in a second range that includes the most recent cervical dilation value included in the third plurality of values, and
each of the second plurality of labeled feature vectors is associated with an indication of one or more unfavorable outcomes experienced by the respective patient;
receive, from the second trained machine learning model, a second output indicative of an updated risk that the patient will experience at least one of the one or more unfavorable outcomes;
generate an updated graph by plotting the second outcome on the graph; and
cause the updated graph to be presented to the user to aid the user in determining whether to recommend intrapartum Cesarean delivery for the patient.

9. The system of claim 8, wherein the range associated with the trained machine learning model includes cervical dilation from about 4 cm to less than 5 cm and the second range associated with the second trained machine learning model includes cervical dilation from about 5 cm to less than 6 cm.

10. A method for predicting a risk of one or more unfavorable labor outcomes in a patient, the method comprising:
generating a feature vector that includes a first plurality of values and a second plurality of values,
wherein the first plurality of values corresponds to a respective plurality of static variables that are knowable at a time a patient goes into labor, and
the second plurality of values corresponds to a respective plurality of dynamic variables that are associated with a particular time during labor, the second plurality of values includes at least a most recent cervical dilation value;
providing the feature vector to a trained machine learning model, wherein the trained machine learning model was trained using a plurality of labeled feature vectors associated with a respective plurality of patients associated with one or more known labor outcomes,
wherein each of the plurality of labeled feature vectors included values corresponding to the plurality of static variables and the plurality of dynamic variables associated with a respective patient and associated with a cervical dilation value in a range that includes the most recent cervical dilation value, and
each of the plurality of labeled feature vectors is associated with an indication of one or more unfavorable outcomes experienced by the respective patient;
receiving, from the trained machine learning model, an output indicative of a risk that the patient will experience at least one of the one or more unfavorable outcomes; and
causing information indicative of the risk to be presented to a user to aid the user in determining whether to recommend intrapartum Cesarean delivery for the patient.

11. The method of claim 10, wherein the trained machine learning model is a gradient boosting machine model comprising a plurality of decision trees.

12. The method of claim 11, further comprising:
receiving, from a baseline machine learning model, a baseline output indicative of a risk that the patient will experience at least one of the one or more unfavorable outcomes based on variables knowable at the time the patient went into labor, wherein the baseline machine learning model was trained using a second plurality of labeled feature vectors associated with a respective plurality of patients associated with one or more known labor outcomes,
wherein each of the second plurality of labeled feature vectors included values corresponding to the plurality of static variables associated with a respective patient and omitted any dynamic variables associated with the respective patient, and
each of the plurality of labeled feature vectors is associated with an indication of one or more unfavorable outcomes experienced by the respective patient; and
wherein generating the feature vector comprises including the baseline output in the feature vector.

13. The method of claim 12, wherein the trained machine learning model is trained to predict risk that the patient will experience at least one of the one or more unfavorable outcomes based on data collected through 4 centimeters (cm) cervical dilation, and
wherein the most recent cervical dilation value is a cervical dilation value that is at least 4 cm cervical dilation and less than 5 cm.

14. The method of claim 10, wherein the plurality of static variables includes variables corresponding to parity, a binary indication of whether the patient has previously delivered via Cesarean, and the patient's age.

15. The method of claim 10, wherein the plurality of dynamic variables includes variables corresponding to cervical dilation, cervical effacement, and head station.

16. The method of claim 10, further comprising:
plotting the outcome on a graph, wherein the graph includes a curve representing average risk scores for patients that did not experience unfavorable outcomes and a second curve representing risk scores for patients that experienced one or more unfavorable outcomes; and
causing the graph to be presented as the information indicative of the risk.

17. The method of claim 16, further comprising:
generating a second feature vector that includes the first plurality of values and a third plurality of values,
wherein the third plurality of values corresponds to a respective plurality of dynamic variables that are associated with a second particular time during labor, including at least a most recent cervical dilation value that exceeds an upper limit of the range associated with the trained model;
providing the second feature vector to a second trained machine learning model, wherein the second machine learning model was trained using a second plurality of labeled feature vectors associated with the respective plurality of patients associated with the one or more known labor outcomes,
wherein each of the second plurality of labeled feature vectors included values corresponding to the plurality of static variables and the plurality of dynamic variables associated with a respective patient and associated with a cervical dilation value in a second range that includes the most recent cervical dilation value included in the third plurality of values, and
each of the second plurality of labeled feature vectors is associated with an indication of one or more unfavorable outcomes experienced by the respective patient;
receiving, from the second trained machine learning model, a second output indicative of an updated risk that the patient will experience at least one of the one or more unfavorable outcomes;
generating an updated graph by plotting the second outcome on the graph; and
causing the updated graph to be presented to the user to aid the user in determining whether to recommend intrapartum Cesarean delivery for the patient.

18. The method of claim 17, wherein the range associated with the trained machine learning model includes cervical dilation from about 4 cm to less than 5 cm and the second range associated with the second trained machine learning model includes cervical dilation from about 5 cm to less than 6 cm.

19. A non-transitory computer readable medium containing computer executable instructions that, when executed by a processor, cause the processor to perform a method for predicting a risk of one or more unfavorable labor outcomes in a patient, the method comprising:
generating a feature vector that includes a first plurality of values and a second plurality of values,
wherein the first plurality of values corresponds to a respective plurality of static variables that are knowable at a time a patient goes into labor, and
the second plurality of values corresponds to a respective plurality of dynamic variables that are associated with a particular time during labor, the second plurality of values includes at least a most recent cervical dilation value;
providing the feature vector to a trained machine learning model, wherein the trained machine learning model was trained using a plurality of labeled feature vectors associated with a respective plurality of patients associated with one or more known labor outcomes,
wherein each of the plurality of labeled feature vectors included values corresponding to the plurality of static variables and the plurality of dynamic variables associated with a respective patient and associated with a cervical dilation value in a range that includes the most recent cervical dilation value, and
each of the plurality of labeled feature vectors is associated with an indication of one or more unfavorable outcomes experienced by the respective patient;
receiving, from the trained machine learning model, an output indicative of a risk that the patient will experience at least one of the one or more unfavorable outcomes; and
causing information indicative of the risk to be presented to a user to aid the user in determining whether to recommend intrapartum Cesarean delivery for the patient.

20-27. (canceled)
